SEQUENCE LISTING (413 sequences)

SEQ ID NO: 1 - RNA, 10 nt, Artificial Sequence
  U1 domain
cagguaagua

SEQ ID NO: 2 - RNA, 10 nt, Artificial Sequence
  U1 snRNA
  misc_RNA (4)...(5): n = pseudouridine
uacnnaccug

SEQ ID NO: 3 - RNA, 10 nt, Artificial Sequence
  U1 domain
gagguaagua

SEQ ID NO: 4 - RNA, 11 nt, Artificial Sequence
  U1 domain
cagguaaguau

SEQ ID NO: 5 - RNA, 13 nt, Artificial Sequence
  U1 domain
gccagguaaguau

SEQ ID NO: 6 - protein, 15 aa, Artificial Sequence
  Tat peptide
YGRKKKRRQRRRPPQ

SEQ ID NO: 7 - protein, 18 aa, Artificial Sequence
  Penetratin
RQIKIWFQNRRMKWKKGG

SEQ ID NO: 8 - DNA, 20 bp, Artificial Sequence
  target site
gcttgtggtgtcattgggat

SEQ ID NO: 9 - RNA, 33 nt, Artificial Sequence
  U1AO sequence
aucccaaugacaccacaagcgccagguaaguau

SEQ ID NO: 10 - RNA, 33 nt, Artificial Sequence
  U1AO sequence
ugcccaagucaucucguuucgccagguaaguau

SEQ ID NO: 11 - DNA, 20 bp, Artificial Sequence
  target sequence
gaaacgagatgacttgggca

SEQ ID NO: 12 - DNA, 20 bp, Artificial Sequence
  target sequence
gtccacgagaaacgagatga

SEQ ID NO: 13 - DNA, 20 bp, Artificial Sequence
  target sequence
tccacgagaaacgagatgac

SEQ ID NO: 14 - DNA, 20 bp, Artificial Sequence
  target sequence
ccacgagaaacgagatgact

SEQ ID NO: 15 - DNA, 20 bp, Artificial Sequence
  target sequence
gatgacttgggcaaaggtga

SEQ ID NO: 16 - DNA, 20 bp, Artificial Sequence
  target sequence
atgacttgggcaaaggtgac

SEQ ID NO: 17 - DNA, 20 bp, Artificial Sequence
  target sequence
tgacttgggcaaaggtgaca

SEQ ID NO: 18 - DNA, 20 bp, Artificial Sequence
  target sequence
acttgggcaaaggtgacaat

SEQ ID NO: 19 - DNA, 20 bp, Artificial Sequence
  target sequence
cttgggcaaaggtgacaatg

SEQ ID NO: 20 - DNA, 20 bp, Artificial Sequence
  target sequence
gcaaaggtgacaatgaagaa

SEQ ID NO: 21 - DNA, 20 bp, Artificial Sequence
  target sequence
ggtgacaatgaagaaagtac

SEQ ID NO: 22 - DNA, 20 bp, Artificial Sequence
  target sequence
gtacacagacaggaaacgcc

SEQ ID NO: 23 - DNA, 20 bp, Artificial Sequence
  target sequence
tacacagacaggaaacgccg

SEQ ID NO: 24 - DNA, 20 bp, Artificial Sequence
  target sequence
acacagacaggaaacgccgg

SEQ ID NO: 25 - DNA, 20 bp, Artificial Sequence
  target sequence
gacaggaaacgccgggagtc

SEQ ID NO: 26 - DNA, 20 bp, Artificial Sequence
  target sequence
gaaacgccgggagtcgtttg

SEQ ID NO: 27 - DNA, 20 bp, Artificial Sequence
  target sequence
aacgccgggagtcgtttggc

SEQ ID NO: 28 - DNA, 20 bp, Artificial Sequence
  target sequence
acgccgggagtcgtttggct

SEQ ID NO: 29 - DNA, 20 bp, Artificial Sequence
  target sequence
cgccgggagtcgtttggctt

SEQ ID NO: 30 - DNA, 20 bp, Artificial Sequence
  target sequence
ggagtcgtttggcttgtggt

SEQ ID NO: 31 - DNA, 20 bp, Artificial Sequence
  target sequence
gagtcgtttggcttgtggtg

SEQ ID NO: 32 - DNA, 20 bp, Artificial Sequence
  target sequence
agtcgtttggcttgtggtgt

SEQ ID NO: 33 - DNA, 20 bp, Artificial Sequence
  target sequence
gtcgtttggcttgtggtgtc

SEQ ID NO: 34 - DNA, 20 bp, Artificial Sequence
  target sequence
tcgtttggcttgtggtgtca

SEQ ID NO: 35 - DNA, 20 bp, Artificial Sequence
  target sequence
cgtttggcttgtggtgtcat

SEQ ID NO: 36 - DNA, 20 bp, Artificial Sequence
  target sequence
tttggcttgtggtgtcattg

SEQ ID NO: 37 - DNA, 20 bp, Artificial Sequence
  target sequence
ttggcttgtggtgtcattgg

SEQ ID NO: 38 - DNA, 20 bp, Artificial Sequence
  target sequence
ggcttgtggtgtcattggga

SEQ ID NO: 39 - DNA, 20 bp, Artificial Sequence
  target sequence
cttgtggtgtcattgggatc

SEQ ID NO: 40 - DNA, 20 bp, Artificial Sequence
  target sequence
gtggtgtcattgggatcgcc

SEQ ID NO: 41 - DNA, 20 bp, Artificial Sequence
  target sequence
tggtgtcattgggatcgcca

SEQ ID NO: 42 - DNA, 20 bp, Artificial Sequence
  target sequence
ggtgtcattgggatcgccaa

SEQ ID NO: 43 - DNA, 20 bp, Artificial Sequence
  target sequence
gtgtcattgggatcgccaag

SEQ ID NO: 44 - DNA, 20 bp, Artificial Sequence
  target sequence
tgtcattgggatcgccaagt

SEQ ID NO: 45 - DNA, 20 bp, Artificial Sequence
  target sequence
tcattgggatcgccaagtaa

SEQ ID NO: 46 - DNA, 20 bp, Artificial Sequence
  target sequence
cattgggatcgccaagtaaa

SEQ ID NO: 47 - DNA, 20 bp, Artificial Sequence
  target sequence
gggatcgccaagtaaatatt

SEQ ID NO: 48 - DNA, 20 bp, Artificial Sequence
  target sequence
ggatcgccaagtaaatattc

SEQ ID NO: 49 - DNA, 20 bp, Artificial Sequence
  target sequence
gatcgccaagtaaatattcc

SEQ ID NO: 50 - DNA, 20 bp, Artificial Sequence
  target sequence
atcgccaagtaaatattccc

SEQ ID NO: 51 - DNA, 20 bp, Artificial Sequence
  target sequence
tcgccaagtaaatattccct

SEQ ID NO: 52 - DNA, 20 bp, Artificial Sequence
  target sequence
ccaagtaaatattccctagg

SEQ ID NO: 53 - DNA, 20 bp, Artificial Sequence
  target sequence
gtaaatattccctaggatgc

SEQ ID NO: 54 - DNA, 20 bp, Artificial Sequence
  target sequence
aatattccctaggatgcgat

SEQ ID NO: 55 - DNA, 20 bp, Artificial Sequence
  target sequence
atattccctaggatgcgatc

SEQ ID NO: 56 - DNA, 20 bp, Artificial Sequence
  target sequence
tattccctaggatgcgatct

SEQ ID NO: 57 - DNA, 20 bp, Artificial Sequence
  target sequence
attccctaggatgcgatctg

SEQ ID NO: 58 - DNA, 20 bp, Artificial Sequence
  target sequence
ttccctaggatgcgatctga

SEQ ID NO: 59 - DNA, 20 bp, Artificial Sequence
  target sequence
tccctaggatgcgatctgag

SEQ ID NO: 60 - DNA, 20 bp, Artificial Sequence
  target sequence
ccctaggatgcgatctgagt

SEQ ID NO: 61 - DNA, 20 bp, Artificial Sequence
  target sequence
ggatgcgatctgagttctag

SEQ ID NO: 62 - DNA, 20 bp, Artificial Sequence
  target sequence
gatgcgatctgagttctagt

SEQ ID NO: 63 - DNA, 20 bp, Artificial Sequence
  target sequence
tgcgatctgagttctagtta

SEQ ID NO: 64 - DNA, 20 bp, Artificial Sequence
  target sequence
cgatctgagttctagttaac

SEQ ID NO: 65 - DNA, 20 bp, Artificial Sequence
  target sequence
tctgagttctagttaactcc

SEQ ID NO: 66 - DNA, 20 bp, Artificial Sequence
  target sequence
ctcctatgttatcttgctag

SEQ ID NO: 67 - DNA, 20 bp, Artificial Sequence
  target sequence
cctatgttatcttgctagtc

SEQ ID NO: 68 - DNA, 20 bp, Artificial Sequence
  target sequence
tgttatcttgctagtcgtag

SEQ ID NO: 69 - DNA, 20 bp, Artificial Sequence
  target sequence
gttatcttgctagtcgtaga

SEQ ID NO: 70 - DNA, 20 bp, Artificial Sequence
  target sequence
agtacctgtaatgagaactg

SEQ ID NO: 71 - DNA, 20 bp, Artificial Sequence
  target sequence
gtacctgtaatgagaactga

SEQ ID NO: 72 - DNA, 20 bp, Artificial Sequence
  target sequence
gtatgtttcaacgatctctg

SEQ ID NO: 73 - DNA, 20 bp, Artificial Sequence
  target sequence
acgatctctgtattttgcca

SEQ ID NO: 74 - DNA, 20 bp, Artificial Sequence
  target sequence
cgatctctgtattttgccag

SEQ ID NO: 75 - DNA, 20 bp, Artificial Sequence
  target sequence
atctctgtattttgccaggc

SEQ ID NO: 76 - DNA, 20 bp, Artificial Sequence
  target sequence
tctctgtattttgccaggct

SEQ ID NO: 77 - DNA, 20 bp, Artificial Sequence
  target sequence
ctgtattttgccaggcttaa

SEQ ID NO: 78 - DNA, 20 bp, Artificial Sequence
  target sequence
gtattttgccaggcttaatc

SEQ ID NO: 79 - DNA, 20 bp, Artificial Sequence
  target sequence
gccaggcttaatcatatatg

SEQ ID NO: 80 - DNA, 20 bp, Artificial Sequence
  target sequence
ccaggcttaatcatatatgg

SEQ ID NO: 81 - DNA, 20 bp, Artificial Sequence
  target sequence
cttcaattctcattcaagcc

SEQ ID NO: 82 - DNA, 20 bp, Artificial Sequence
  target sequence
tcaattctcattcaagcccg

SEQ ID NO: 83 - DNA, 20 bp, Artificial Sequence
  target sequence
caattctcattcaagcccgc

SEQ ID NO: 84 - DNA, 20 bp, Artificial Sequence
  target sequence
attctcattcaagcccgcaa

SEQ ID NO: 85 - DNA, 20 bp, Artificial Sequence
  target sequence
ttctcattcaagcccgcaat

SEQ ID NO: 86 - DNA, 20 bp, Artificial Sequence
  target sequence
tctcattcaagcccgcaata

SEQ ID NO: 87 - DNA, 20 bp, Artificial Sequence
  target sequence
ctcattcaagcccgcaataa

SEQ ID NO: 88 - DNA, 20 bp, Artificial Sequence
  target sequence
cattcaagcccgcaataaac

SEQ ID NO: 89 - DNA, 20 bp, Artificial Sequence
  target sequence
ttcaagcccgcaataaacaa

SEQ ID NO: 90 - DNA, 20 bp, Artificial Sequence
  target sequence
tcaagcccgcaataaacaaa

SEQ ID NO: 91 - DNA, 20 bp, Artificial Sequence
  target sequence
caagcccgcaataaacaaat

SEQ ID NO: 92 - DNA, 20 bp, Artificial Sequence
  target sequence
tggcactgaattcttaacct

SEQ ID NO: 93 - DNA, 20 bp, Artificial Sequence
  target sequence
ggcactgaattcttaaccta

SEQ ID NO: 94 - DNA, 33 bp, Artificial Sequence
  U1AO sequence (DNA format)
tcatctcgtttctcgtggacgccaggtaagtat

SEQ ID NO: 95 - DNA, 33 bp, Artificial Sequence
  U1AO sequence (DNA format)
gtcatctcgtttctcgtggagccaggtaagtat

SEQ ID NO: 96 - DNA, 33 bp, Artificial Sequence
  U1AO sequence (DNA format)
agtcatctcgtttctcgtgggccaggtaagtat

SEQ ID NO: 97 - DNA, 33 bp, Artificial Sequence
  U1AO sequence (DNA format)
tgcccaagtcatctcgtttcgccaggtaagtat

SEQ ID NO: 98 - DNA, 33 bp, Artificial Sequence
  U1AO sequence (DNA format)
tcacctttgcccaagtcatcgccaggtaagtat

SEQ ID NO: 99 - DNA, 33 bp, Artificial Sequence
  U1AO sequence (DNA format)
gtcacctttgcccaagtcatgccaggtaagtat

SEQ ID NO: 100 - DNA, 33 bp, Artificial Sequence
  U1AO sequence (DNA format)
tgtcacctttgcccaagtcagccaggtaagtat

SEQ ID NO: 101 - DNA, 33 bp, Artificial Sequence
  U1AO sequence (DNA format)
attgtcacctttgcccaagtgccaggtaagtat

SEQ ID NO: 102 - DNA, 33 bp, Artificial Sequence
  U1AO sequence (DNA format)
cattgtcacctttgcccaaggccaggtaagtat

SEQ ID NO: 103 - DNA, 33 bp, Artificial Sequence
  U1AO sequence (DNA format)
ttcttcattgtcacctttgcgccaggtaagtat

SEQ ID NO: 104 - DNA, 33 bp, Artificial Sequence
  U1AO sequence (DNA format)
gtactttcttcattgtcaccgccaggtaagtat

SEQ ID NO: 105 - DNA, 33 bp, Artificial Sequence
  U1AO sequence (DNA format)
ggcgtttcctgtctgtgtacgccaggtaagtat

SEQ ID NO: 106 - DNA, 33 bp, Artificial Sequence
  U1AO sequence (DNA format)
cggcgtttcctgtctgtgtagccaggtaagtat

SEQ ID NO: 107 - DNA, 33 bp, Artificial Sequence
  U1AO sequence (DNA format)
ccggcgtttcctgtctgtgtgccaggtaagtat

SEQ ID NO: 108 - DNA, 33 bp, Artificial Sequence
  U1AO sequence (DNA format)
gactcccggcgtttcctgtcgccaggtaagtat

SEQ ID NO: 109 - DNA, 33 bp, Artificial Sequence
  U1AO sequence (DNA format)
caaacgactcccggcgtttcgccaggtaagtat

SEQ ID NO: 110 - DNA, 33 bp, Artificial Sequence
  U1AO sequence (DNA format)
gccaaacgactcccggcgttgccaggtaagtat

SEQ ID NO: 111 - DNA, 33 bp, Artificial Sequence
  U1AO sequence (DNA format)
agccaaacgactcccggcgtgccaggtaagtat

SEQ ID NO: 112 - DNA, 33 bp, Artificial Sequence
  U1AO sequence (DNA format)
aagccaaacgactcccggcggccaggtaagtat

SEQ ID NO: 113 - DNA, 33 bp, Artificial Sequence
  U1AO sequence (DNA format)
accacaagccaaacgactccgccaggtaagtat

SEQ ID NO: 114 - DNA, 33 bp, Artificial Sequence
  U1AO sequence (DNA format)
caccacaagccaaacgactcgccaggtaagtat

SEQ ID NO: 115 - DNA, 33 bp, Artificial Sequence
  U1AO sequence (DNA format)
acaccacaagccaaacgactgccaggtaagtat

SEQ ID NO: 116 - DNA, 33 bp, Artificial Sequence
  U1AO sequence (DNA format)
gacaccacaagccaaacgacgccaggtaagtat

SEQ ID NO: 117 - DNA, 33 bp, Artificial Sequence
  U1AO sequence (DNA format)
tgacaccacaagccaaacgagccaggtaagtat

SEQ ID NO: 118 - DNA, 33 bp, Artificial Sequence
  U1AO sequence (DNA format)
atgacaccacaagccaaacggccaggtaagtat

SEQ ID NO: 119 - DNA, 33 bp, Artificial Sequence
  U1AO sequence (DNA format)
caatgacaccacaagccaaagccaggtaagtat

SEQ ID NO: 120 - DNA, 33 bp, Artificial Sequence
  U1AO sequence (DNA format)
ccaatgacaccacaagccaagccaggtaagtat

SEQ ID NO: 121 - DNA, 33 bp, Artificial Sequence
  U1AO sequence (DNA format)
tcccaatgacaccacaagccgccaggtaagtat

SEQ ID NO: 122 - DNA, 33 bp, Artificial Sequence
  U1AO sequence (DNA format)
atcccaatgacaccacaagcgccaggtaagtat

SEQ ID NO: 123 - DNA, 33 bp, Artificial Sequence
  U1AO sequence (DNA format)
gatcccaatgacaccacaaggccaggtaagtat

SEQ ID NO: 124 - DNA, 33 bp, Artificial Sequence
  U1AO sequence (DNA format)
ggcgatcccaatgacaccacgccaggtaagtat

SEQ ID NO: 125 - DNA, 33 bp, Artificial Sequence
  U1AO sequence (DNA format)
tggcgatcccaatgacaccagccaggtaagtat

SEQ ID NO: 126 - DNA, 33 bp, Artificial Sequence
  U1AO sequence (DNA format)
ttggcgatcccaatgacaccgccaggtaagtat

SEQ ID NO: 127 - DNA, 33 bp, Artificial Sequence
  U1AO sequence (DNA format)
cttggcgatcccaatgacacgccaggtaagtat

SEQ ID NO: 128 - DNA, 33 bp, Artificial Sequence
  U1AO sequence (DNA format)
acttggcgatcccaatgacagccaggtaagtat

SEQ ID NO: 129 - DNA, 33 bp, Artificial Sequence
  U1AO sequence (DNA format)
ttacttggcgatcccaatgagccaggtaagtat

SEQ ID NO: 130 - DNA, 33 bp, Artificial Sequence
  U1AO sequence (DNA format)
tttacttggcgatcccaatggccaggtaagtat

SEQ ID NO: 131 - DNA, 33 bp, Artificial Sequence
  U1AO sequence (DNA format)
aatatttacttggcgatcccgccaggtaagtat

SEQ ID NO: 132 - DNA, 33 bp, Artificial Sequence
  U1AO sequence (DNA format)
gaatatttacttggcgatccgccaggtaagtat

SEQ ID NO: 133 - DNA, 33 bp, Artificial Sequence
  U1AO sequence (DNA format)
ggaatatttacttggcgatcgccaggtaagtat

SEQ ID NO: 134 - DNA, 33 bp, Artificial Sequence
  U1AO sequence (DNA format)
gggaatatttacttggcgatgccaggtaagtat

SEQ ID NO: 135 - DNA, 33 bp, Artificial Sequence
  U1AO sequence (DNA format)
agggaatatttacttggcgagccaggtaagtat

SEQ ID NO: 136 - DNA, 33 bp, Artificial Sequence
  U1AO sequence (DNA format)
cctagggaatatttacttgggccaggtaagtat

SEQ ID NO: 137 - DNA, 33 bp, Artificial Sequence
  U1AO sequence (DNA format)
gcatcctagggaatatttacgccaggtaagtat

SEQ ID NO: 138 - DNA, 33 bp, Artificial Sequence
  U1AO sequence (DNA format)
atcgcatcctagggaatattgccaggtaagtat

SEQ ID NO: 139 - DNA, 33 bp, Artificial Sequence
  U1AO sequence (DNA format)
gatcgcatcctagggaatatgccaggtaagtat

SEQ ID NO: 140 - DNA, 33 bp, Artificial Sequence
  U1AO sequence (DNA format)
agatcgcatcctagggaatagccaggtaagtat

SEQ ID NO: 141 - DNA, 33 bp, Artificial Sequence
  U1AO sequence (DNA format)
cagatcgcatcctagggaatgccaggtaagtat

SEQ ID NO: 142 - DNA, 33 bp, Artificial Sequence
  U1AO sequence (DNA format)
tcagatcgcatcctagggaagccaggtaagtat

SEQ ID NO: 143 - DNA, 33 bp, Artificial Sequence
  U1AO sequence (DNA format)
ctcagatcgcatcctagggagccaggtaagtat

SEQ ID NO: 144 - DNA, 33 bp, Artificial Sequence
  U1AO sequence (DNA format)
actcagatcgcatcctaggggccaggtaagtat

SEQ ID NO: 145 - DNA, 33 bp, Artificial Sequence
  U1AO sequence (DNA format)
ctagaactcagatcgcatccgccaggtaagtat

SEQ ID NO: 146 - DNA, 33 bp, Artificial Sequence
  U1AO sequence (DNA format)
actagaactcagatcgcatcgccaggtaagtat

SEQ ID NO: 147 - DNA, 33 bp, Artificial Sequence
  U1AO sequence (DNA format)
taactagaactcagatcgcagccaggtaagtat

SEQ ID NO: 148 - DNA, 33 bp, Artificial Sequence
  U1AO sequence (DNA format)
gttaactagaactcagatcggccaggtaagtat

SEQ ID NO: 149 - DNA, 33 bp, Artificial Sequence
  U1AO sequence (DNA format)
ggagttaactagaactcagagccaggtaagtat

SEQ ID NO: 150 - DNA, 33 bp, Artificial Sequence
  U1AO sequence (DNA format)
ctagcaagataacataggaggccaggtaagtat

SEQ ID NO: 151 - DNA, 33 bp, Artificial Sequence
  U1AO sequence (DNA format)
gactagcaagataacatagggccaggtaagtat

SEQ ID NO: 152 - DNA, 33 bp, Artificial Sequence
  U1AO sequence (DNA format)
ctacgactagcaagataacagccaggtaagtat

SEQ ID NO: 153 - DNA, 33 bp, Artificial Sequence
  U1AO sequence (DNA format)
tctacgactagcaagataacgccaggtaagtat

SEQ ID NO: 154 - DNA, 33 bp, Artificial Sequence
  U1AO sequence (DNA format)
cagttctcattacaggtactgccaggtaagtat

SEQ ID NO: 155 - DNA, 33 bp, Artificial Sequence
  U1AO sequence (DNA format)
tcagttctcattacaggtacgccaggtaagtat

SEQ ID NO: 156 - DNA, 33 bp, Artificial Sequence
  U1AO sequence (DNA format)
cagagatcgttgaaacatacgccaggtaagtat

SEQ ID NO: 157 - DNA, 33 bp, Artificial Sequence
  U1AO sequence (DNA format)
tggcaaaatacagagatcgtgccaggtaagtat

SEQ ID NO: 158 - DNA, 33 bp, Artificial Sequence
  U1AO sequence (DNA format)
ctggcaaaatacagagatcggccaggtaagtat

SEQ ID NO: 159 - DNA, 33 bp, Artificial Sequence
  U1AO sequence (DNA format)
gcctggcaaaatacagagatgccaggtaagtat

SEQ ID NO: 160 - DNA, 33 bp, Artificial Sequence
  U1AO sequence (DNA format)
agcctggcaaaatacagagagccaggtaagtat

SEQ ID NO: 161 - DNA, 33 bp, Artificial Sequence
  U1AO sequence (DNA format)
ttaagcctggcaaaatacaggccaggtaagtat

SEQ ID NO: 162 - DNA, 33 bp, Artificial Sequence
  U1AO sequence (DNA format)
gattaagcctggcaaaatacgccaggtaagtat

SEQ ID NO: 163 - DNA, 33 bp, Artificial Sequence
  U1AO sequence (DNA format)
catatatgattaagcctggcgccaggtaagtat

SEQ ID NO: 164 - DNA, 33 bp, Artificial Sequence
  U1AO sequence (DNA format)
ccatatatgattaagcctgggccaggtaagtat

SEQ ID NO: 165 - DNA, 33 bp, Artificial Sequence
  U1AO sequence (DNA format)
ggcttgaatgagaattgaaggccaggtaagtat

SEQ ID NO: 166 - DNA, 33 bp, Artificial Sequence
  U1AO sequence (DNA format)
cgggcttgaatgagaattgagccaggtaagtat

SEQ ID NO: 167 - DNA, 33 bp, Artificial Sequence
  U1AO sequence (DNA format)
gcgggcttgaatgagaattggccaggtaagtat

SEQ ID NO: 168 - DNA, 33 bp, Artificial Sequence
  U1AO sequence (DNA format)
ttgcgggcttgaatgagaatgccaggtaagtat

SEQ ID NO: 169 - DNA, 33 bp, Artificial Sequence
  U1AO sequence (DNA format)
attgcgggcttgaatgagaagccaggtaagtat

SEQ ID NO: 170 - DNA, 33 bp, Artificial Sequence
  U1AO sequence (DNA format)
tattgcgggcttgaatgagagccaggtaagtat

SEQ ID NO: 171 - DNA, 33 bp, Artificial Sequence
  U1AO sequence (DNA format)
ttattgcgggcttgaatgaggccaggtaagtat

SEQ ID NO: 172 - DNA, 33 bp, Artificial Sequence
  U1AO sequence (DNA format)
gtttattgcgggcttgaatggccaggtaagtat

SEQ ID NO: 173 - DNA, 33 bp, Artificial Sequence
  U1AO sequence (DNA format)
ttgtttattgcgggcttgaagccaggtaagtat

SEQ ID NO: 174 - DNA, 33 bp, Artificial Sequence
  U1AO sequence (DNA format)
tttgtttattgcgggcttgagccaggtaagtat

SEQ ID NO: 175 - DNA, 33 bp, Artificial Sequence
  U1AO sequence (DNA format)
atttgtttattgcgggcttggccaggtaagtat

SEQ ID NO: 176 - DNA, 33 bp, Artificial Sequence
  U1AO sequence (DNA format)
aggttaagaattcagtgccagccaggtaagtat

SEQ ID NO: 177 - DNA, 33 bp, Artificial Sequence
  U1AO sequence (DNA format)
taggttaagaattcagtgccgccaggtaagtat

SEQ ID NO: 178 - DNA, 20 bp, Artificial Sequence
  target sequence
gtccatgaaaaagcagatga

SEQ ID NO: 179 - DNA, 20 bp, Artificial Sequence
  target sequence
tccatgaaaaagcagatgac

SEQ ID NO: 180 - DNA, 20 bp, Artificial Sequence
  target sequence
ccatgaaaaagcagatgact

SEQ ID NO: 181 - DNA, 20 bp, Artificial Sequence
  target sequence
gcagatgacttgggcaaagg

SEQ ID NO: 182 - DNA, 20 bp, Artificial Sequence
  target sequence
cagatgacttgggcaaaggt

SEQ ID NO: 183 - DNA, 20 bp, Artificial Sequence
  target sequence
gatgacttgggcaaaggtgg

SEQ ID NO: 184 - DNA, 20 bp, Artificial Sequence
  target sequence
atgacttgggcaaaggtgga

SEQ ID NO: 185 - DNA, 20 bp, Artificial Sequence
  target sequence
tgacttgggcaaaggtggaa

SEQ ID NO: 186 - DNA, 20 bp, Artificial Sequence
  target sequence
acttgggcaaaggtggaaat

SEQ ID NO: 187 - DNA, 20 bp, Artificial Sequence
  target sequence
cttgggcaaaggtggaaatg

SEQ ID NO: 188 - DNA, 20 bp, Artificial Sequence
  target sequence
gcaaaggtggaaatgaagaa

SEQ ID NO: 189 - DNA, 20 bp, Artificial Sequence
  target sequence
gtacaaagacaggaaacgct

SEQ ID NO: 190 - DNA, 20 bp, Artificial Sequence
  target sequence
gacaggaaacgctggaagtc

SEQ ID NO: 191 - DNA, 20 bp, Artificial Sequence
  target sequence
gaaacgctggaagtcgtttg

SEQ ID NO: 192 - DNA, 20 bp, Artificial Sequence
  target sequence
aacgctggaagtcgtttggc

SEQ ID NO: 193 - DNA, 20 bp, Artificial Sequence
  target sequence
acgctggaagtcgtttggct

SEQ ID NO: 194 - DNA, 20 bp, Artificial Sequence
  target sequence
cgctggaagtcgtttggctt

SEQ ID NO: 195 - DNA, 20 bp, Artificial Sequence
  target sequence
gaagtcgtttggcttgtggt

SEQ ID NO: 196 - DNA, 20 bp, Artificial Sequence
  target sequence
aagtcgtttggcttgtggtg

SEQ ID NO: 197 - DNA, 20 bp, Artificial Sequence
  target sequence
agtcgtttggcttgtggtgt

SEQ ID NO: 198 - DNA, 20 bp, Artificial Sequence
  target sequence
gtcgtttggcttgtggtgta

SEQ ID NO: 199 - DNA, 20 bp, Artificial Sequence
  target sequence
tcgtttggcttgtggtgtaa

SEQ ID NO: 200 - DNA, 20 bp, Artificial Sequence
  target sequence
cgtttggcttgtggtgtaat

SEQ ID NO: 201 - DNA, 20 bp, Artificial Sequence
  target sequence
tttggcttgtggtgtaattg

SEQ ID NO: 202 - DNA, 20 bp, Artificial Sequence
  target sequence
ttggcttgtggtgtaattgg

SEQ ID NO: 203 - DNA, 20 bp, Artificial Sequence
  target sequence
ggcttgtggtgtaattggga

SEQ ID NO: 204 - DNA, 20 bp, Artificial Sequence
  target sequence
gcttgtggtgtaattgggat

SEQ ID NO: 205 - DNA, 20 bp, Artificial Sequence
  target sequence
cttgtggtgtaattgggatc

SEQ ID NO: 206 - DNA, 20 bp, Artificial Sequence
  target sequence
gtggtgtaattgggatcgcc

SEQ ID NO: 207 - DNA, 20 bp, Artificial Sequence
  target sequence
tggtgtaattgggatcgccc

SEQ ID NO: 208 - DNA, 20 bp, Artificial Sequence
  target sequence
ggtgtaattgggatcgccca

SEQ ID NO: 209 - DNA, 20 bp, Artificial Sequence
  target sequence
gtgtaattgggatcgcccaa

SEQ ID NO: 210 - DNA, 20 bp, Artificial Sequence
  target sequence
tgtaattgggatcgcccaat

SEQ ID NO: 211 - DNA, 20 bp, Artificial Sequence
  target sequence
taattgggatcgcccaataa

SEQ ID NO: 212 - DNA, 20 bp, Artificial Sequence
  target sequence
aattgggatcgcccaataaa

SEQ ID NO: 213 - DNA, 20 bp, Artificial Sequence
  target sequence
attgggatcgcccaataaac

SEQ ID NO: 214 - DNA, 20 bp, Artificial Sequence
  target sequence
gggatcgcccaataaacatt

SEQ ID NO: 215 - DNA, 20 bp, Artificial Sequence
  target sequence
ggatcgcccaataaacattc

SEQ ID NO: 216 - DNA, 20 bp, Artificial Sequence
  target sequence
gatcgcccaataaacattcc

SEQ ID NO: 217 - DNA, 20 bp, Artificial Sequence
  target sequence
atcgcccaataaacattccc

SEQ ID NO: 218 - DNA, 20 bp, Artificial Sequence
  target sequence
tcgcccaataaacattccct

SEQ ID NO: 219 - DNA, 20 bp, Artificial Sequence
  target sequence
cccaataaacattcccttgg

SEQ ID NO: 220 - DNA, 20 bp, Artificial Sequence
  target sequence
aacattcccttggatgtagt

SEQ ID NO: 221 - DNA, 20 bp, Artificial Sequence
  target sequence
acattcccttggatgtagtc

SEQ ID NO: 222 - DNA, 20 bp, Artificial Sequence
  target sequence
cattcccttggatgtagtct

SEQ ID NO: 223 - DNA, 20 bp, Artificial Sequence
  target sequence
ttcccttggatgtagtctga

SEQ ID NO: 224 - DNA, 20 bp, Artificial Sequence
  target sequence
tcccttggatgtagtctgag

SEQ ID NO: 225 - DNA, 20 bp, Artificial Sequence
  target sequence
cccttggatgtagtctgagg

SEQ ID NO: 226 - DNA, 20 bp, Artificial Sequence
  target sequence
tggatgtagtctgaggcccc

SEQ ID NO: 227 - DNA, 20 bp, Artificial Sequence
  target sequence
ggatgtagtctgaggcccct

SEQ ID NO: 228 - DNA, 20 bp, Artificial Sequence
  target sequence
gatgtagtctgaggcccctt

SEQ ID NO: 229 - DNA, 20 bp, Artificial Sequence
  target sequence
atgtagtctgaggcccctta

SEQ ID NO: 230 - DNA, 20 bp, Artificial Sequence
  target sequence
tgtagtctgaggccccttaa

SEQ ID NO: 231 - DNA, 20 bp, Artificial Sequence
  target sequence
tagtctgaggccccttaact

SEQ ID NO: 232 - DNA, 20 bp, Artificial Sequence
  target sequence
agtctgaggccccttaactc

SEQ ID NO: 233 - DNA, 20 bp, Artificial Sequence
  target sequence
gtctgaggccccttaactca

SEQ ID NO: 234 - DNA, 20 bp, Artificial Sequence
  target sequence
tctgaggccccttaactcat

SEQ ID NO: 235 - DNA, 20 bp, Artificial Sequence
  target sequence
ctgaggccccttaactcatc

SEQ ID NO: 236 - DNA, 20 bp, Artificial Sequence
  target sequence
gaggccccttaactcatctg

SEQ ID NO: 237 - DNA, 20 bp, Artificial Sequence
  target sequence
aggccccttaactcatctgt

SEQ ID NO: 238 - DNA, 20 bp, Artificial Sequence
  target sequence
ggccccttaactcatctgtt

SEQ ID NO: 239 - DNA, 20 bp, Artificial Sequence
  target sequence
gccccttaactcatctgtta

SEQ ID NO: 240 - DNA, 20 bp, Artificial Sequence
  target sequence
ccccttaactcatctgttat

SEQ ID NO: 241 - DNA, 20 bp, Artificial Sequence
  target sequence
taactcatctgttatcctgc

SEQ ID NO: 242 - DNA, 20 bp, Artificial Sequence
  target sequence
aactcatctgttatcctgct

SEQ ID NO: 243 - DNA, 20 bp, Artificial Sequence
  target sequence
actcatctgttatcctgcta

SEQ ID NO: 244 - DNA, 20 bp, Artificial Sequence
  target sequence
ctcatctgttatcctgctag

SEQ ID NO: 245 - DNA, 20 bp, Artificial Sequence
  target sequence
catctgttatcctgctagct

SEQ ID NO: 246 - DNA, 20 bp, Artificial Sequence
  target sequence
tctgttatcctgctagctgt

SEQ ID NO: 247 - DNA, 20 bp, Artificial Sequence
  target sequence
ctgttatcctgctagctgta

SEQ ID NO: 248 - DNA, 20 bp, Artificial Sequence
  target sequence
gttatcctgctagctgtaga

SEQ ID NO: 249 - DNA, 20 bp, Artificial Sequence
  target sequence
ttatcctgctagctgtagaa

SEQ ID NO: 250 - DNA, 20 bp, Artificial Sequence
  target sequence
tatcctgctagctgtagaaa

SEQ ID NO: 251 - DNA, 20 bp, Artificial Sequence
  target sequence
tcctgctagctgtagaaatg

SEQ ID NO: 252 - DNA, 20 bp, Artificial Sequence
  target sequence
cctgctagctgtagaaatgt

SEQ ID NO: 253 - DNA, 20 bp, Artificial Sequence
  target sequence
gctagctgtagaaatgtatc

SEQ ID NO: 254 - DNA, 20 bp, Artificial Sequence
  target sequence
ctagctgtagaaatgtatcc

SEQ ID NO: 255 - DNA, 20 bp, Artificial Sequence
  target sequence
gctgtagaaatgtatcctga

SEQ ID NO: 256 - DNA, 20 bp, Artificial Sequence
  target sequence
gtgtgactttttcagagttg

SEQ ID NO: 257 - DNA, 20 bp, Artificial Sequence
  target sequence
tgtgactttttcagagttgc

SEQ ID NO: 258 - DNA, 20 bp, Artificial Sequence
  target sequence
gtgactttttcagagttgct

SEQ ID NO: 259 - DNA, 20 bp, Artificial Sequence
  target sequence
gagttgctttaaagtacctg

SEQ ID NO: 260 - DNA, 20 bp, Artificial Sequence
  target sequence
agtacctgtagtgagaaact

SEQ ID NO: 261 - DNA, 20 bp, Artificial Sequence
  target sequence
gtacctgtagtgagaaactg

SEQ ID NO: 262 - DNA, 20 bp, Artificial Sequence
  target sequence
tacctgtagtgagaaactga

SEQ ID NO: 263 - DNA, 20 bp, Artificial Sequence
  target sequence
acctgtagtgagaaactgat

SEQ ID NO: 264 - DNA, 20 bp, Artificial Sequence
  target sequence
cctgtagtgagaaactgatt

SEQ ID NO: 265 - DNA, 20 bp, Artificial Sequence
  target sequence
atgtctgtttcaatgacctg

SEQ ID NO: 266 - DNA, 20 bp, Artificial Sequence
  target sequence
tgtctgtttcaatgacctgt

SEQ ID NO: 267 - DNA, 20 bp, Artificial Sequence
  target sequence
gtctgtttcaatgacctgta

SEQ ID NO: 268 - DNA, 20 bp, Artificial Sequence
  target sequence
atgacctgtattttgccaga

SEQ ID NO: 269 - DNA, 20 bp, Artificial Sequence
  target sequence
tgacctgtattttgccagac

SEQ ID NO: 270 - DNA, 20 bp, Artificial Sequence
  target sequence
gacctgtattttgccagact

SEQ ID NO: 271 - DNA, 20 bp, Artificial Sequence
  target sequence
acctgtattttgccagactt

SEQ ID NO: 272 - DNA, 20 bp, Artificial Sequence
  target sequence
cctgtattttgccagactta

SEQ ID NO: 273 - DNA, 20 bp, Artificial Sequence
  target sequence
ttgccagacttaaatcacag

SEQ ID NO: 274 - DNA, 20 bp, Artificial Sequence
  target sequence
tgccagacttaaatcacaga

SEQ ID NO: 275 - DNA, 20 bp, Artificial Sequence
  target sequence
gccagacttaaatcacagat

SEQ ID NO: 276 - DNA, 20 bp, Artificial Sequence
  target sequence
ccagacttaaatcacagatg

SEQ ID NO: 277 - DNA, 20 bp, Artificial Sequence
  target sequence
gacttaaatcacagatgggt

SEQ ID NO: 278 - DNA, 20 bp, Artificial Sequence
  target sequence
ttctttgtcattcaagcctg

SEQ ID NO: 279 - DNA, 20 bp, Artificial Sequence
  target sequence
tctttgtcattcaagcctgt

SEQ ID NO: 280 - DNA, 20 bp, Artificial Sequence
  target sequence
ctttgtcattcaagcctgtg

SEQ ID NO: 281 - DNA, 20 bp, Artificial Sequence
  target sequence
ttgtcattcaagcctgtgaa

SEQ ID NO: 282 - DNA, 20 bp, Artificial Sequence
  target sequence
tgtcattcaagcctgtgaat

SEQ ID NO: 283 - DNA, 20 bp, Artificial Sequence
  target sequence
gtcattcaagcctgtgaata

SEQ ID NO: 284 - DNA, 20 bp, Artificial Sequence
  target sequence
caagcctgtgaataaaaacc

SEQ ID NO: 285 - DNA, 20 bp, Artificial Sequence
  target sequence
agcctgtgaataaaaaccct

SEQ ID NO: 286 - DNA, 20 bp, Artificial Sequence
  target sequence
gcctgtgaataaaaaccctg

SEQ ID NO: 287 - DNA, 20 bp, Artificial Sequence
  target sequence
cctgtgaataaaaaccctgt

SEQ ID NO: 288 - DNA, 20 bp, Artificial Sequence
  target sequence
aaaccctgtatggcacttat

SEQ ID NO: 289 - DNA, 20 bp, Artificial Sequence
  target sequence
aaccctgtatggcacttatt

SEQ ID NO: 290 - DNA, 20 bp, Artificial Sequence
  target sequence
accctgtatggcacttatta

SEQ ID NO: 291 - DNA, 20 bp, Artificial Sequence
  target sequence
ccctgtatggcacttattat

SEQ ID NO: 292 - DNA, 20 bp, Artificial Sequence
  target sequence
gtatggcacttattatgagg

SEQ ID NO: 293 - DNA, 20 bp, Artificial Sequence
  target sequence
tatggcacttattatgaggc

SEQ ID NO: 294 - DNA, 20 bp, Artificial Sequence
  target sequence
tggcacttattatgaggcta

SEQ ID NO: 295 - DNA, 20 bp, Artificial Sequence
  target sequence
ggcacttattatgaggctat

SEQ ID NO: 296 - DNA, 33 bp, Artificial Sequence
  U1AO sequence (DNA format)
tcatctgctttttcatggacgccaggtaagtat

SEQ ID NO: 297 - DNA, 33 bp, Artificial Sequence
  U1AO sequence (DNA format)
gtcatctgctttttcatggagccaggtaagtat

SEQ ID NO: 298 - DNA, 33 bp, Artificial Sequence
  U1AO sequence (DNA format)
agtcatctgctttttcatgggccaggtaagtat

SEQ ID NO: 299 - DNA, 33 bp, Artificial Sequence
  U1AO sequence (DNA format)
cctttgcccaagtcatctgcgccaggtaagtat

SEQ ID NO: 300 - DNA, 33 bp, Artificial Sequence
  U1AO sequence (DNA format)
acctttgcccaagtcatctggccaggtaagtat

SEQ ID NO: 301 - DNA, 33 bp, Artificial Sequence
  U1AO sequence (DNA format)
ccacctttgcccaagtcatcgccaggtaagtat

SEQ ID NO: 302 - DNA, 33 bp, Artificial Sequence
  U1AO sequence (DNA format)
tccacctttgcccaagtcatgccaggtaagtat

SEQ ID NO: 303 - DNA, 33 bp, Artificial Sequence
  U1AO sequence (DNA format)
ttccacctttgcccaagtcagccaggtaagtat

SEQ ID NO: 304 - DNA, 33 bp, Artificial Sequence
  U1AO sequence (DNA format)
atttccacctttgcccaagtgccaggtaagtat

SEQ ID NO: 305 - DNA, 33 bp, Artificial Sequence
  U1AO sequence (DNA format)
catttccacctttgcccaaggccaggtaagtat

SEQ ID NO: 306 - DNA, 33 bp, Artificial Sequence
  U1AO sequence (DNA format)
ttcttcatttccacctttgcgccaggtaagtat

SEQ ID NO: 307 - DNA, 33 bp, Artificial Sequence
  U1AO sequence (DNA format)
agcgtttcctgtctttgtacgccaggtaagtat

SEQ ID NO: 308 - DNA, 33 bp, Artificial Sequence
  U1AO sequence (DNA format)
gacttccagcgtttcctgtcgccaggtaagtat

SEQ ID NO: 309 - DNA, 33 bp, Artificial Sequence
  U1AO sequence (DNA format)
caaacgacttccagcgtttcgccaggtaagtat

SEQ ID NO: 310 - DNA, 33 bp, Artificial Sequence
  U1AO sequence (DNA format)
gccaaacgacttccagcgttgccaggtaagtat

SEQ ID NO: 311 - DNA, 33 bp, Artificial Sequence
  U1AO sequence (DNA format)
agccaaacgacttccagcgtgccaggtaagtat

SEQ ID NO: 312 - DNA, 33 bp, Artificial Sequence
  U1AO sequence (DNA format)
aagccaaacgacttccagcggccaggtaagtat

SEQ ID NO: 313 - DNA, 33 bp, Artificial Sequence
  U1AO sequence (DNA format)
accacaagccaaacgacttcgccaggtaagtat

SEQ ID NO: 314 - DNA, 33 bp, Artificial Sequence
  U1AO sequence (DNA format)
caccacaagccaaacgacttgccaggtaagtat

SEQ ID NO: 315 - DNA, 33 bp, Artificial Sequence; identical to SEQ ID NO: 115
  U1AO sequence (DNA format)

SEQ ID NO: 316 - DNA, 33 bp, Artificial Sequence
  U1AO sequence (DNA format)
tacaccacaagccaaacgacgccaggtaagtat

SEQ ID NO: 317 - DNA, 33 bp, Artificial Sequence
  U1AO sequence (DNA format)
ttacaccacaagccaaacgagccaggtaagtat

SEQ ID NO: 318 - DNA, 33 bp, Artificial Sequence
  U1AO sequence (DNA format)
attacaccacaagccaaacggccaggtaagtat

SEQ ID NO: 319 - DNA, 33 bp, Artificial Sequence
  U1AO sequence (DNA format)
caattacaccacaagccaaagccaggtaagtat

SEQ ID NO: 320 - DNA, 33 bp, Artificial Sequence
  U1AO sequence (DNA format)
ccaattacaccacaagccaagccaggtaagtat

SEQ ID NO: 321 - DNA, 33 bp, Artificial Sequence
  U1AO sequence (DNA format)
tcccaattacaccacaagccgccaggtaagtat

SEQ ID NO: 322 - DNA, 33 bp, Artificial Sequence
  U1AO sequence (DNA format)
atcccaattacaccacaagcgccaggtaagtat

SEQ ID NO: 323 - DNA, 33 bp, Artificial Sequence
  U1AO sequence (DNA format)
gatcccaattacaccacaaggccaggtaagtat

SEQ ID NO: 324 - DNA, 33 bp, Artificial Sequence
  U1AO sequence (DNA format)
ggcgatcccaattacaccacgccaggtaagtat

SEQ ID NO: 325 - DNA, 33 bp, Artificial Sequence
  U1AO sequence (DNA format)
gggcgatcccaattacaccagccaggtaagtat

SEQ ID NO: 326 - DNA, 33 bp, Artificial Sequence
  U1AO sequence (DNA format)
tgggcgatcccaattacaccgccaggtaagtat

SEQ ID NO: 327 - DNA, 33 bp, Artificial Sequence
  U1AO sequence (DNA format)
ttgggcgatcccaattacacgccaggtaagtat

SEQ ID NO: 328 - DNA, 33 bp, Artificial Sequence
  U1AO sequence (DNA format)
attgggcgatcccaattacagccaggtaagtat

SEQ ID NO: 329 - DNA, 33 bp, Artificial Sequence
  U1AO sequence (DNA format)
ttattgggcgatcccaattagccaggtaagtat

SEQ ID NO: 330 - DNA, 33 bp, Artificial Sequence
  U1AO sequence (DNA format)
tttattgggcgatcccaattgccaggtaagtat

SEQ ID NO: 331 - DNA, 33 bp, Artificial Sequence
  U1AO sequence (DNA format)
gtttattgggcgatcccaatgccaggtaagtat

SEQ ID NO: 332 - DNA, 33 bp, Artificial Sequence
  U1AO sequence (DNA format)
aatgtttattgggcgatcccgccaggtaagtat

SEQ ID NO: 333 - DNA, 33 bp, Artificial Sequence
  U1AO sequence (DNA format)
gaatgtttattgggcgatccgccaggtaagtat

SEQ ID NO: 334 - DNA, 33 bp, Artificial Sequence
  U1AO sequence (DNA format)
ggaatgtttattgggcgatcgccaggtaagtat

SEQ ID NO: 335 - DNA, 33 bp, Artificial Sequence
  U1AO sequence (DNA format)
gggaatgtttattgggcgatgccaggtaagtat

SEQ ID NO: 336 - DNA, 33 bp, Artificial Sequence
  U1AO sequence (DNA format)
agggaatgtttattgggcgagccaggtaagtat

SEQ ID NO: 337 - DNA, 33 bp, Artificial Sequence
  U1AO sequence (DNA format)
ccaagggaatgtttattggggccaggtaagtat

SEQ ID NO: 338 - DNA, 33 bp, Artificial Sequence
  U1AO sequence (DNA format)
actacatccaagggaatgttgccaggtaagtat

SEQ ID NO: 339 - DNA, 33 bp, Artificial Sequence
  U1AO sequence (DNA format)
gactacatccaagggaatgtgccaggtaagtat

SEQ ID NO: 340 - DNA, 33 bp, Artificial Sequence
  U1AO sequence (DNA format)
agactacatccaagggaatggccaggtaagtat

SEQ ID NO: 341 - DNA, 33 bp, Artificial Sequence
  U1AO sequence (DNA format)
tcagactacatccaagggaagccaggtaagtat

SEQ ID NO: 342 - DNA, 33 bp, Artificial Sequence
  U1AO sequence (DNA format)
ctcagactacatccaagggagccaggtaagtat

SEQ ID NO: 343 - DNA, 33 bp, Artificial Sequence
  U1AO sequence (DNA format)
cctcagactacatccaaggggccaggtaagtat

SEQ ID NO: 344 - DNA, 33 bp, Artificial Sequence
  U1AO sequence (DNA format)
ggggcctcagactacatccagccaggtaagtat

SEQ ID NO: 345 - DNA, 33 bp, Artificial Sequence
  U1AO sequence (DNA format)
aggggcctcagactacatccgccaggtaagtat

SEQ ID NO: 346 - DNA, 33 bp, Artificial Sequence
  U1AO sequence (DNA format)
aaggggcctcagactacatcgccaggtaagtat

SEQ ID NO: 347 - DNA, 33 bp, Artificial Sequence
  U1AO sequence (DNA format)
taaggggcctcagactacatgccaggtaagtat

SEQ ID NO: 348 - DNA, 33 bp, Artificial Sequence
  U1AO sequence (DNA format)
ttaaggggcctcagactacagccaggtaagtat

SEQ ID NO: 349 - DNA, 33 bp, Artificial Sequence
  U1AO sequence (DNA format)
agttaaggggcctcagactagccaggtaagtat

SEQ ID NO: 350 - DNA, 33 bp, Artificial Sequence
  U1AO sequence (DNA format)
gagttaaggggcctcagactgccaggtaagtat

SEQ ID NO: 351 - DNA, 33 bp, Artificial Sequence
  U1AO sequence (DNA format)
tgagttaaggggcctcagacgccaggtaagtat

SEQ ID NO: 352 - DNA, 33 bp, Artificial Sequence
  U1AO sequence (DNA format)
atgagttaaggggcctcagagccaggtaagtat

SEQ ID NO: 353 - DNA, 33 bp, Artificial Sequence
  U1AO sequence (DNA format)
gatgagttaaggggcctcaggccaggtaagtat

SEQ ID NO: 354 - DNA, 33 bp, Artificial Sequence
  U1AO sequence (DNA format)
cagatgagttaaggggcctcgccaggtaagtat

SEQ ID NO: 355 - DNA, 33 bp, Artificial Sequence
  U1AO sequence (DNA format)
acagatgagttaaggggcctgccaggtaagtat

SEQ ID NO: 356 - DNA, 33 bp, Artificial Sequence
  U1AO sequence (DNA format)
aacagatgagttaaggggccgccaggtaagtat

SEQ ID NO: 357 - DNA, 33 bp, Artificial Sequence
  U1AO sequence (DNA format)
taacagatgagttaaggggcgccaggtaagtat

SEQ ID NO: 358 - DNA, 33 bp, Artificial Sequence
  U1AO sequence (DNA format)
ataacagatgagttaagggggccaggtaagtat

SEQ ID NO: 359 - DNA, 33 bp, Artificial Sequence
  U1AO sequence (DNA format)
gcaggataacagatgagttagccaggtaagtat

SEQ ID NO: 360 - DNA, 33 bp, Artificial Sequence
  U1AO sequence (DNA format)
agcaggataacagatgagttgccaggtaagtat

SEQ ID NO: 361 - DNA, 33 bp, Artificial Sequence
  U1AO sequence (DNA format)
tagcaggataacagatgagtgccaggtaagtat

SEQ ID NO: 362 - DNA, 33 bp, Artificial Sequence
  U1AO sequence (DNA format)
ctagcaggataacagatgaggccaggtaagtat

SEQ ID NO: 363 - DNA, 33 bp, Artificial Sequence
  U1AO sequence (DNA format)
agctagcaggataacagatggccaggtaagtat

SEQ ID NO: 364 - DNA, 33 bp, Artificial Sequence
  U1AO sequence (DNA format)
acagctagcaggataacagagccaggtaagtat

SEQ ID NO: 365 - DNA, 33 bp, Artificial Sequence
  U1AO sequence (DNA format)
tacagctagcaggataacaggccaggtaagtat

SEQ ID NO: 366 - DNA, 33 bp, Artificial Sequence
  U1AO sequence (DNA format)
tctacagctagcaggataacgccaggtaagtat

SEQ ID NO: 367 - DNA, 33 bp, Artificial Sequence
  U1AO sequence (DNA format)
ttctacagctagcaggataagccaggtaagtat

SEQ ID NO: 368 - DNA, 33 bp, Artificial Sequence
  U1AO sequence (DNA format)
tttctacagctagcaggatagccaggtaagtat

SEQ ID NO: 369 - DNA, 33 bp, Artificial Sequence
  U1AO sequence (DNA format)
catttctacagctagcaggagccaggtaagtat

SEQ ID NO: 370 - DNA, 33 bp, Artificial Sequence
  U1AO sequence (DNA format)
acatttctacagctagcagggccaggtaagtat

SEQ ID NO: 371 - DNA, 33 bp, Artificial Sequence
  U1AO sequence (DNA format)
gatacatttctacagctagcgccaggtaagtat

SEQ ID NO: 372 - DNA, 33 bp, Artificial Sequence
  U1AO sequence (DNA format)
ggatacatttctacagctaggccaggtaagtat

SEQ ID NO: 373 - DNA, 33 bp, Artificial Sequence
  U1AO sequence (DNA format)
tcaggatacatttctacagcgccaggtaagtat

SEQ ID NO: 374 - DNA, 33 bp, Artificial Sequence
  U1AO sequence (DNA format)
caactctgaaaaagtcacacgccaggtaagtat

SEQ ID NO: 375 - DNA, 33 bp, Artificial Sequence
  U1AO sequence (DNA format)
gcaactctgaaaaagtcacagccaggtaagtat

SEQ ID NO: 376 - DNA, 33 bp, Artificial Sequence
  U1AO sequence (DNA format)
agcaactctgaaaaagtcacgccaggtaagtat

SEQ ID NO: 377 - DNA, 33 bp, Artificial Sequence
  U1AO sequence (DNA format)
caggtactttaaagcaactcgccaggtaagtat

SEQ ID NO: 378 - DNA, 33 bp, Artificial Sequence
  U1AO sequence (DNA format)
agtttctcactacaggtactgccaggtaagtat

SEQ ID NO: 379 - DNA, 33 bp, Artificial Sequence
  U1AO sequence (DNA format)
cagtttctcactacaggtacgccaggtaagtat

SEQ ID NO: 380 - DNA, 33 bp, Artificial Sequence
  U1AO sequence (DNA format)
tcagtttctcactacaggtagccaggtaagtat

SEQ ID NO: 381 - DNA, 33 bp, Artificial Sequence
  U1AO sequence (DNA format)
atcagtttctcactacaggtgccaggtaagtat

SEQ ID NO: 382 - DNA, 33 bp, Artificial Sequence
  U1AO sequence (DNA format)
aatcagtttctcactacagggccaggtaagtat

SEQ ID NO: 383 - DNA, 33 bp, Artificial Sequence
  U1AO sequence (DNA format)
caggtcattgaaacagacatgccaggtaagtat

SEQ ID NO: 384 - DNA, 33 bp, Artificial Sequence
  U1AO sequence (DNA format)
acaggtcattgaaacagacagccaggtaagtat

SEQ ID NO: 385 - DNA, 33 bp, Artificial Sequence
  U1AO sequence (DNA format)
tacaggtcattgaaacagacgccaggtaagtat

SEQ ID NO: 386 - DNA, 33 bp, Artificial Sequence
  U1AO sequence (DNA format)
tctggcaaaatacaggtcatgccaggtaagtat

SEQ ID NO: 387 - DNA, 33 bp, Artificial Sequence
  U1AO sequence (DNA format)
gtctggcaaaatacaggtcagccaggtaagtat

SEQ ID NO: 388 - DNA, 33 bp, Artificial Sequence
  U1AO sequence (DNA format)
agtctggcaaaatacaggtcgccaggtaagtat

SEQ ID NO: 389 - DNA, 33 bp, Artificial Sequence
  U1AO sequence (DNA format)
aagtctggcaaaatacaggtgccaggtaagtat

SEQ ID NO: 390 - DNA, 33 bp, Artificial Sequence
  U1AO sequence (DNA format)
taagtctggcaaaatacagggccaggtaagtat

SEQ ID NO: 391 - DNA, 33 bp, Artificial Sequence
  U1AO sequence (DNA format)
ctgtgatttaagtctggcaagccaggtaagtat

SEQ ID NO: 392 - DNA, 33 bp, Artificial Sequence
  U1AO sequence (DNA format)
tctgtgatttaagtctggcagccaggtaagtat

SEQ ID NO: 393 - DNA, 33 bp, Artificial Sequence
  U1AO sequence (DNA format)
atctgtgatttaagtctggcgccaggtaagtat

SEQ ID NO: 394 - DNA, 33 bp, Artificial Sequence
  U1AO sequence (DNA format)
catctgtgatttaagtctgggccaggtaagtat

SEQ ID NO: 395 - DNA, 33 bp, Artificial Sequence
  U1AO sequence (DNA format)
acccatctgtgatttaagtcgccaggtaagtat

SEQ ID NO: 396 - DNA, 33 bp, Artificial Sequence
  U1AO sequence (DNA format)
caggcttgaatgacaaagaagccaggtaagtat

SEQ ID NO: 397 - DNA, 33 bp, Artificial Sequence
  U1AO sequence (DNA format)
acaggcttgaatgacaaagagccaggtaagtat

SEQ ID NO: 398 - DNA, 33 bp, Artificial Sequence
  U1AO sequence (DNA format)
cacaggcttgaatgacaaaggccaggtaagtat

SEQ ID NO: 399 - DNA, 33 bp, Artificial Sequence
  U1AO sequence (DNA format)
ttcacaggcttgaatgacaagccaggtaagtat

SEQ ID NO: 400 - DNA, 33 bp, Artificial Sequence
  U1AO sequence (DNA format)
attcacaggcttgaatgacagccaggtaagtat

SEQ ID NO: 401 - DNA, 33 bp, Artificial Sequence
  U1AO sequence (DNA format)
tattcacaggcttgaatgacgccaggtaagtat

SEQ ID NO: 402 - DNA, 33 bp, Artificial Sequence
  U1AO sequence (DNA format)
ggtttttattcacaggcttggccaggtaagtat

SEQ ID NO: 403 - DNA, 33 bp, Artificial Sequence
  U1AO sequence (DNA format)
agggtttttattcacaggctgccaggtaagtat

SEQ ID NO: 404 - DNA, 33 bp, Artificial Sequence
  U1AO sequence (DNA format)
cagggtttttattcacaggcgccaggtaagtat

SEQ ID NO: 405 - DNA, 33 bp, Artificial Sequence
  U1AO sequence (DNA format)
acagggtttttattcacagggccaggtaagtat

SEQ ID NO: 406 - DNA, 33 bp, Artificial Sequence
  U1AO sequence (DNA format)
ataagtgccatacagggtttgccaggtaagtat

SEQ ID NO: 407 - DNA, 33 bp, Artificial Sequence
  U1AO sequence (DNA format)
aataagtgccatacagggttgccaggtaagtat

SEQ ID NO: 408 - DNA, 33 bp, Artificial Sequence
  U1AO sequence (DNA format)
taataagtgccatacagggtgccaggtaagtat

SEQ ID NO: 409 - DNA, 33 bp, Artificial Sequence
  U1AO sequence (DNA format)
ataataagtgccatacaggggccaggtaagtat

SEQ ID NO: 410 - DNA, 33 bp, Artificial Sequence
  U1AO sequence (DNA format)
cctcataataagtgccatacgccaggtaagtat

SEQ ID NO: 411 - DNA, 33 bp, Artificial Sequence
  U1AO sequence (DNA format)
gcctcataataagtgccatagccaggtaagtat

SEQ ID NO: 412 - DNA, 33 bp, Artificial Sequence
  U1AO sequence (DNA format)
tagcctcataataagtgccagccaggtaagtat

SEQ ID NO: 413 - DNA, 33 bp, Artificial Sequence
  U1AO sequence (DNA format)
atagcctcataataagtgccgccaggtaagtat

(19) United States
(12) Patent Application Publication
(10) Pub. No.: US 2022/0372488 A1
(43) Pub. Date: Nov. 24, 2022

(54) COMPOSITIONS AND METHODS FOR TREATING NEURODEGENERATIVE DISORDERS

(71) Applicants: RUTGERS, THE STATE UNIVERSITY OF NEW JERSEY, NEW BRUNSWICK, NJ (US); SILAGENE, INC., HILLSBOROUGH, NJ (US)

(72) Inventors: SAMUEL IAN GUNDERSON, PISCATAWAY, NJ (US); RAFAL GORACZNIAK, HILLSBOROUGH, NJ (US)

(21) Appl. No.: 17/441,905

(22) PCT Filed: Mar. 25, 2020
(86) PCT No.: PCT/US2020/024554

§ 371 Date: Sep. 22, 2021

Related U.S. Application Data
(60) Provisional application No. 62/824,066, filed on Mar. 26, 2019.

(51) Int. Cl. C12N 15/113; A61P 25/00

(52) U.S. Cl. CPC C12N 15/1137; A61P 25/00; C12N 2310/321; C12N 2310/315; C12N 2320/31

(57) ABSTRACT

Compositions and methods are provided for the inhibition, treatment and/or prevention of degenerative myelopathy (DM) or amyotrophic lateral sclerosis (ALS).

### CROSS REFERENCE TO RELATED APPLICATIONS

This application is a U.S. National Stage Application under 35 U.S.C. § 371 of International Application No. PCT/US2020/024554, filed Mar. 25, 2020, which claims priority under 35 U.S.C. § 119(e) to U.S. Provisional Patent Application No. 62/824,066, filed Mar. 26, 2019, the entire contents of each of which are hereby incorporated by reference herein.

### SEQUENCE LISTING

The instant specification contains a Sequence Listing which has been submitted in ASCII format via EFS-Web and is hereby incorporated by reference in its entirety. Said ASCII copy, created on May 16, 2022, is named “721292_SLG-100US_REPLACEMENT_SEQUENCE_LISTING” and is 73.8 KB in size.

### FIELD OF THE INVENTION

This invention relates generally to the field of gene silencing. Specifically, the invention provides compositions and methods for regulating the expression of the superoxide dismutase gene.

### BACKGROUND OF THE INVENTION

Degenerative myelopathy (DM) is a slowly progressive spinal cord disorder that closely resembles Amyotrophic Lateral Sclerosis (ALS or Lou Gehrig's Disease) in humans. Specifically, DM is a neurodegenerative disease of old dogs that affects many breeds and causes paralysis over a period of 6-12 months (Averill, D. R., J. Am. Vet. Med. Assoc. (1973) 162(12):1045-1051; Coates, et al., Vet. Clin. North Am. Small Anim. Prac. (2010) 40(5):929-950; Coates, et al., J. Vet. Intern. Med. (2007) 21(6):1323-1331; Shelton, et al., J. Neurol. Sci. (2012) 318(1-2):55-64; Braund, et al., Am. J. Vet. Res. (1978) 39(8):1309-1315). DM is highly associated with a mutation in the gene for superoxide dismutase, SOD1. The mutation does not cause a loss of function, but causes misfolding and aggregation of the SOD1 protein, resulting in neuronal death (Kohyama, et al., J. Vet. Med. Sci. (2017) 79(2): 375-379; Nakamae, et al., Neuroscience (2015) 303:229-40; Crisp et al., Exp. Neurol. (2013), 248:1-9; Turner, et al., Prog. Neurobiol. (2008) 85:94-134). Similarly, mutant SOD1 protein leads to toxic aggregates in ALS (Tiwari, et al. Neurodegener. Dis. (2005) 2(3-4):115-27; Rakhit, et al., Biochim. Biophys. Acta. (2006) 1762(11-12):1025-37; Benkler et al., Sci. Rep. (2018) 8(1):16393). Reducing expression of this mutated gene therefore represents an important therapeutic target.

### SUMMARY OF THE INVENTION

In accordance with the instant invention, nucleic acid molecules for inhibiting the expression of the superoxide dismutase 1 gene (SOD) are provided. In a particular embodiment, the nucleic acid molecules comprise an annealing domain operably linked to at least one effector domain, wherein the annealing domain hybridizes to the pre-mRNA of SOD (e.g., canine or human) and wherein the effector domain hybridizes to the U1 snRNA of U1 snRNP.

In accordance with another aspect of the instant invention, the nucleic acid molecules may be conjugated to (e.g., directly or via a linker) a targeting moiety. The targeting moiety may be conjugated to any chemically feasible location including internal nucleotides, the 5′ end and/or the 3′ end (e.g., the nucleic acid may comprise two targeting moieties, either the same or different). In a particular embodiment, the nucleic acid molecules are conjugated to an aptamer.

In accordance with another aspect of the invention, methods are provided for inhibiting the expression of SOD comprising delivering to a cell at least one of the nucleic acid molecules of the instant invention.

In accordance with another aspect of the invention, compositions are provided which comprise at least one of the nucleic acid molecules of the invention and at least one pharmaceutically acceptable carrier.

In still another aspect, vectors encoding the nucleic acid molecules of the instant invention are also provided.

In accordance with another aspect of the instant invention, methods of treating, inhibiting, and/or preventing a neurodegenerative disease (e.g., degenerative myelopathy (DM) or amyotrophic lateral sclerosis (ALS)) in a subject are provided. The methods comprise administering a therapeutically effective amount of at least one nucleic acid molecule of the instant invention (e.g., U1 AO or vector encoding the U1AO) to a subject in need thereof. In a particular embodiment, the method comprises administering more than one U1AO.

### BRIEF DESCRIPTIONS OF THE DRAWING

FIG. 1A is a schematic of a U1 adaptor oligonucleotide depicting its 2 domains: an annealing domain to base pair to the target gene's pre-mRNA in the 3′ terminal exon and an effector domain that inhibits maturation of the pre-mRNA via binding of endogenous U1 snRNP. The provided sequence of the effector domain is SEQ ID NO: 1. FIG. 1B is a schematic of the U1 adaptor annealing to target pre-mRNA. The provided sequence of the effector domain is SEQ ID NO: 1. FIG. 1C is a schematic of the U1 adaptor binding U1 snRNP, which leads to poly(A) site inhibition. Ψ=pseudouridines of the U1 snRNA in the U1 snRNP. The provided sequence of the U1 snRNA in the U1 snRNP is SEQ ID NO: 2.

FIG. 2 provides a graph of a quantitative reverse transcription PCR (RT-qPCR) analysis on MDCK cells for canine SOD1. cDNA amounts are 50 ng, 25 ng, 12.5 ng, 6.2 ng, 3.1 ng, and 1.5 ng.

FIG. 3 provides a graph showing the expression of canine SOD1 mRNA in MDCK cells that were untreated or treated with 20 nM control U1AO, 5 nM cSOD1-1, or 20 nM cSOD1-2. Canine SOD1 mRNA expression in untreated MDCK cells was set to 100%. Expression was normalized to canine HPRT1.

FIG. 4 provides a graph showing the expression of canine SOD1 mRNA in MDCK cells that were untreated or treated with 20 nM control U1AO or the indicated amounts of cSOD1-1 or cSOD1-2. Canine SOD1 mRNA expression in untreated MDCK cells was set to 100%. Expression was normalized to canine HPRT1.

FIG. 5 provides a graph showing the expression of canine SOD1 mRNA in MDCK cells that were untreated or treated with the indicated amounts of control U1AO, cSOD1-1 or cSOD1-2. Canine SOD1 mRNA expression in untreated MDCK cells was set to 100%. Expression was normalized to canine HPRT1.

FIG. 6 provides a graph showing the expression of canine SOD1 mRNA in different regions of the central nervous system and spinal cord regions of dogs treated with saline (control treated, n=2) or cSOD1-1 (n=4). Canine SOD1 mRNA expression in saline treated dogs was set to 100%. Expression was normalized to canine HPRT1. Cbl: cerebellum; Ctx: parietal cortex; Hpc: hippocampus; Str: striatum; SC: spinal cord; T: thoracic; C: cervical; L: lumbar.

FIGS. 7A and 7B provide target sites in canine SOD1 for U1AO and examples of U1AO sequences in DNA format. The target sequences in rows 4 and 29 are SEQ ID NOs: 11 and 8, respectively. The target sequences in rows 1-3, 5-28, and 30-84 are SEQ ID NOs: 12-93, respectively. The U1AO sequences provided in DNA format are SEQ ID Nos: 94-177, from top to bottom.

FIGS. 8A-8C provide target sites in human SOD1 for U1AO and examples of U1AO sequences in DNA format. The target sequences are SEQ ID NOs: 178-295, from top to bottom. The U1AO sequences provided in DNA format are SEQ ID Nos: 296-413, from top to bottom.

FIG. 9 provides the pharmacokinetic/pharmacodynamic (PK/PD) data for Dog-73A. The top graph shows the level of expression of SOD1 normalized to HPRT in different regions of the CNS and spinal cord. Saline treated dog set to 100%. The bottom graph shows the amount of U1AO per total RNA in different regions of the CNS and spinal cord. Cbl: cerebellum; Ctx: parietal cortex; Hpc: hippocampus; Str: striatum; SC: spinal cord; T: thoracic; C: cervical; L: lumbar; ROB: rest of brain.

FIG. 10 provides the pharmacokinetic/pharmacodynamic (PK/PD) data for Dog-73E. The top graph shows the level of expression of SOD1 normalized to HPRT in different regions of the CNS and spinal cord. Saline treated dog set to 100%. The bottom graph shows the amount of U1AO per total RNA in different regions of the CNS and spinal cord. Cbl: cerebellum; Ctx: parietal cortex; Hpc: hippocampus; Str: striatum; SC: spinal cord; T: thoracic; C: cervical; L: lumbar; ROB: rest of brain.

FIG. 11 provides the pharmacokinetic/pharmacodynamic (PK/PD) data for Dog-73C undergoing a single IT treatment with 5 day duration. The top graph shows the level of expression of SOD1 normalized to HPRT in different regions of the CNS and spinal cord. Saline treated dog set to 100%. The bottom graph shows the amount of U1AO per total RNA in different regions of the CNS and spinal cord. Cbl: cerebellum; Ctx: parietal cortex; Hpc: hippocampus; Str: striatum; SC: spinal cord; T: thoracic; C: cervical; L: lumbar; ROB: rest of brain.

### DETAILED DESCRIPTION OF THE INVENTION

U1 Adaptors (or U1 adaptor oligonucleotides (U1AO)) are an oligonucleotide-mediated gene silencing technology which are mechanistically distinct from antisense or siRNA. U1 Adaptors act by selectively interfering with a key step in mRNA maturation: the addition of a 3′ polyadenosine (polyA) tail. Nearly all protein-coding mRNAs require a polyA tail and the failure to add one results in rapid degradation of the nascent mRNA inside the nucleus, thereby preventing expression of a protein product. U1 Adaptors have been described in U.S. Pat. Nos. 9,441,221; 9,078,823; 8,907,075; and 8,343,941 (each of which is incorporated by reference herein). U1 Adaptor oligonucleotides are well suited to in vivo applications because they can accept extensive chemical modifications to improve nuclease resistance and the attachment of bulky groups, such as tags for imaging or ligands for receptor-mediated uptake by target cells, without loss of silencing activity.

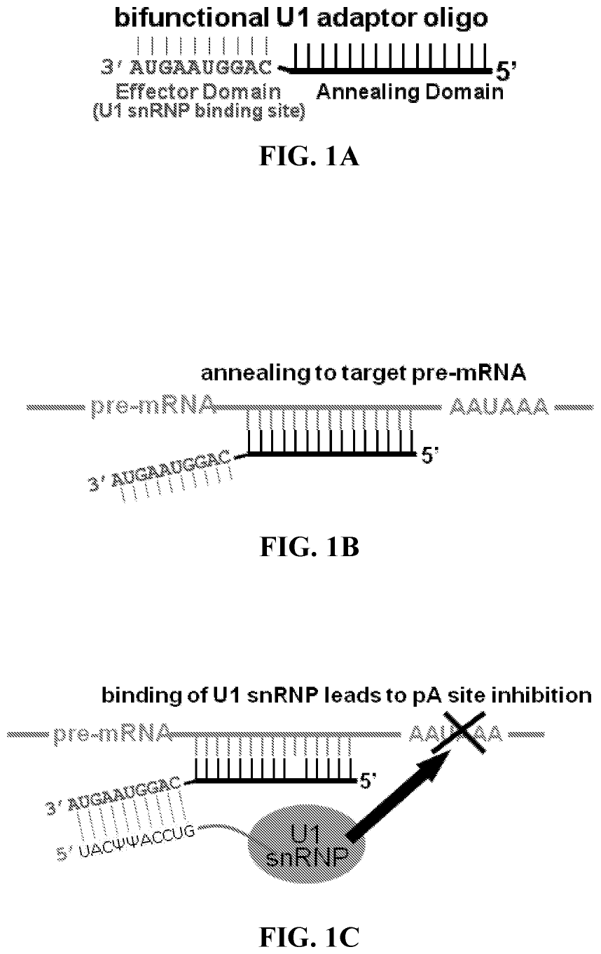

Provided herein are methods and compositions for the modulation of the expression of SOD, particularly SOD1 (e.g., human or canine). The methods comprise the use of a U1 adaptor oligonucleotide/molecule (see, generally, FIG. 1). In its simplest form, the U1AO is an oligonucleotide with two domains: (1) an annealing domain designed to base pair to the SOD gene's pre-mRNA (e.g., in the terminal exon) and (2) an effector domain (also referred to as the U1 domain) that inhibits 3′-end formation of the target pre-mRNA via binding endogenous U1 snRNP. Without being bound by theory, the U1 adaptor tethers endogenous U1 snRNP to a gene-specific pre-mRNA and the resulting complex blocks proper 3′ end formation. Notably, U1 snRNP is highly abundant (˜1 million/mammalian cell nucleus) and in stoichiometric excess compared to other spliceosome components. Therefore, there are no deleterious effects of titrating out endogenous U1 snRNP.

The U1AO is able to enter cells either alone or in complex with delivery reagents (e.g., lipid-based transfection reagents). The U1AO is also capable of entering the nucleus to bind to pre-mRNA. Indeed, this property has already been established for small nucleic acid molecules such as in those antisense approaches that utilize the RNase H pathway where the oligo enters the nucleus and binds to pre-mRNA. Additionally, it has been shown that antisense oligos can bind to nuclear pre-mRNA and sterically block access of splicing factors leading to altered splicing patterns (Ittig et al. (2004) Nuc. Acids Res., 32:346-53).

In a particular embodiment, the annealing domain of the U1 adaptor molecule is designed to have high affinity and specificity to the target site on the target pre-mRNA (e.g., to the exclusion of other pre-mRNAs). In a particular embodiment, a balance should be achieved between having the annealing domain too short, as this will jeopardize affinity, or too long, as this will promote “off-target” effects or alter other cellular pathways. Furthermore, the annealing domain should not interfere with the function of the effector domain (for example, by base pairing and hairpin formation). The U1AO annealing domain does not have an absolute requirement on length. However, the annealing domain will typically be from about 10 to about 50 nucleotides in length, more typically from about 10 to about 30 nucleotides or about 10 to about 20 nucleotides. In a particular embodiment, the annealing domain is at least about 13 or 15 nucleotides in length. The annealing domain may be at least 75%, at least 80%, at least 85%, at least 90%, at least 95%, at least 97%, or, more particularly, 100% complementary to the gene of interest (SOD (e.g., canine or human)). In one embodiment, the annealing domain hybridizes with a target site within the 3′ terminal exon, which includes the terminal coding region and the 3′UTR and polyadenylation signal sequences (e.g., through the polyadenylation site). In another embodiment, the target sequence is within about 500 basepair, about 250 basepair, about 100 basepair, or about 50 bp of the poly(A) signal sequence.

Exemplary amino acid and nucleotide sequences of canine SOD1 can be found, for example, in Gene ID: 403559 and GenBank Accession Nos. NM_001003035.1 and NP_001003035. Exemplary amino acid and nucleotide sequences of human SOD1 can be found, for example, in Gene ID: 6647 and GenBank Accession Nos. NM_000454 and NP_000445.1.

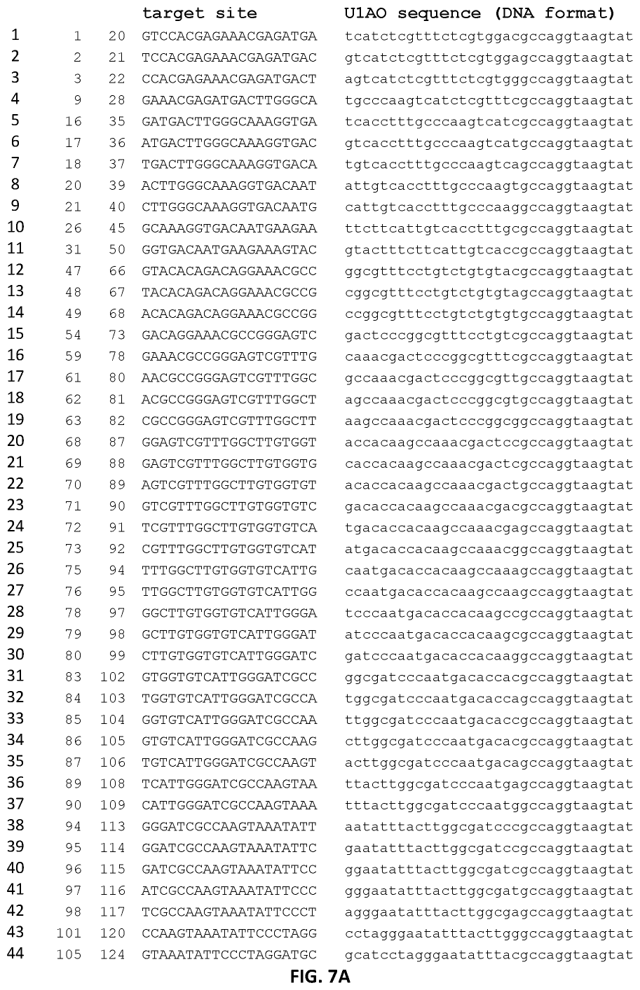

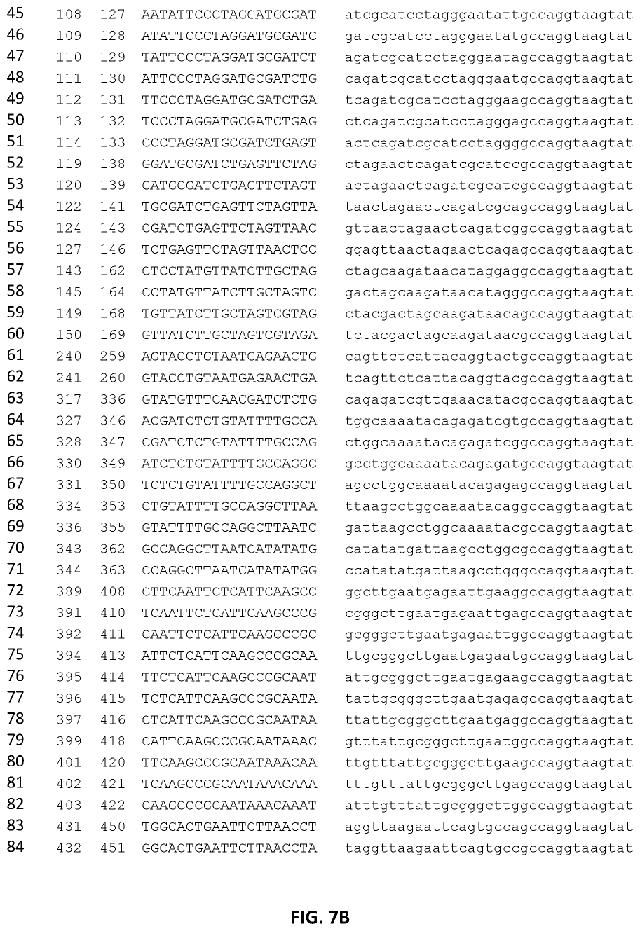

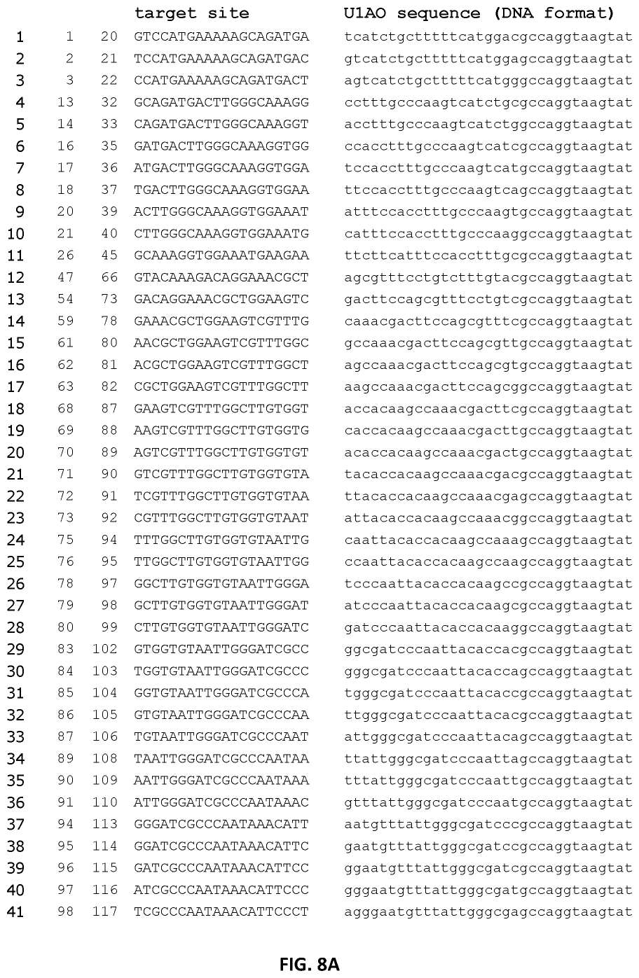

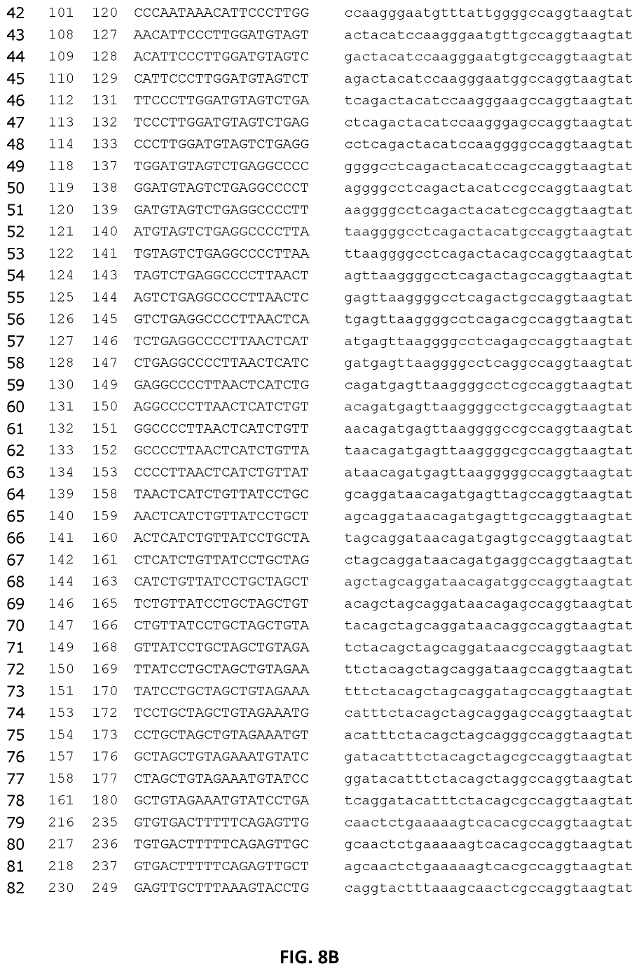

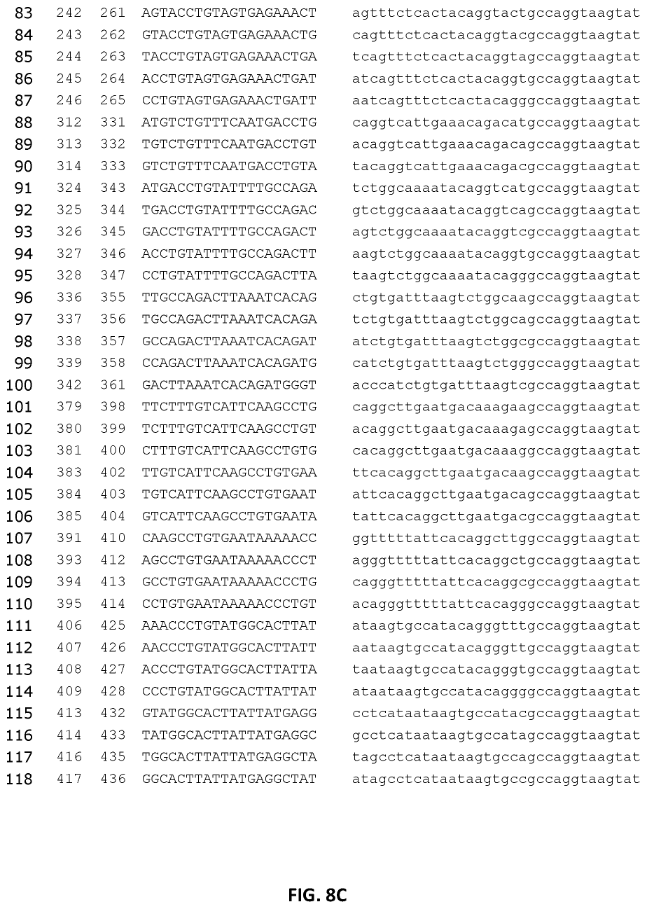

Target sites within SOD for the U1AO have been identified herein using selection criteria for gene silencing. FIGS. 7A and 7B list target sites within canine SOD for the U1AO with the best scoring target sites. In a particular embodiment, the annealing domain hybridizes with a target site provided in FIGS. 7A-7B. FIGS. 8A-8C list target sites within human SOD for the U1AO with the best scoring target sites. In a particular embodiment, the annealing domain hybridizes with a target site provided in FIGS. 8A-8C. In a particular embodiment, the annealing domain hybridizes with GCTTGTGGTGTCATTGGGAT (SEQ ID NO: 8) or GAAACGAGATGACTTGGGCA (SEQ ID NO: 11). The annealing domain may be at least 75%, at least 80%, at least 85%, at least 90%, at least 95%, at least 97%, or, more particularly, 100% complementary to any target sequence within FIG. 7A-7B or 8A-8C or SEQ ID NO: 8 or SEQ ID NO: 11, particularly SEQ ID NO: 8. The annealing domain may comprise additional or fewer nucleotides at the 5′ and/or 3′ end of any target sequence within FIG. 7A-7B or 8A-8C or SEQ ID NO: 8 or SEQ ID NO: 11, particularly SEQ ID NO: 8. For example, the annealing domain may comprise at least 1, 2, 3, 4, 5, or up to 10 or 20 nucleotides added to the 5′ and/or 3′ end of any target sequence within FIG. 7A-7B or 8A-8C or SEQ ID NO: 8 or SEQ ID NO: 11, particularly SEQ ID NO: 8 (e.g., from the sequence of the SOD gene) or may have a deletion of at least 1, 2, 3, 4, or 5 nucleotides from the 5′ and/or 3′ end of any target sequence within FIG. 7A-7B or 8A-8C or SEQ ID NO: 8 or SEQ ID NO: 11, particularly SEQ ID NO: 8.

In a particular embodiment, the U1 domain of the U1 AO binds with high affinity to U1 snRNP. In a particular embodiment, the U1 domain is complementary to nucleotides 2-11 of endogenous U1 snRNA. In a particular embodiment, the U1 domain comprises 5′-CAGGUAAGUA-3′ (SEQ ID NO: 1); 5′-CAGGUAAGUAU-3′ (SEQ ID NO: 4); 5′-GCCAGGUAAGUAU-3′ (SEQ ID NO: 5). In a particular embodiment, the U1 domain comprises the sequence 5′-CAGGUAAGUA-3′ (SEQ ID NO: 1). In a particular embodiment, the U1 domain comprises the sequence 5′-GCCAGGUAAGUAU-3′ (SEQ ID NO: 5). In another embodiment, the U1 domain has at least 70%, at least 75%, at least 80%, at least 85%, and more particularly at least 90%, at least 95%, or at least 97% identity to SEQ ID NO: 1, SEQ ID NO: 4, or SEQ ID NO: 5. The U1 domain may comprise additional nucleotides 5′ or 3′ to SEQ ID NO: 1, SEQ ID NO: 4, or SEQ ID NO: 5. For example, the U1 domain may comprise at least 1, 2, 3, 4, 5, or up to 10 or 20 nucleotides 5′ or 3′ to SEQ ID NO: 1, SEQ ID NO: 4, or SEQ ID NO: 5. Indeed, increasing the length of the U1 domain to include basepairing into stem 1 and/or basepairing to position 1 of U1 snRNA improves the U1 adaptor's affinity to U1 snRNP. The effector domain may be from about 8 nucleotides to about 30 nucleotides, from about 10 nucleotides to about 20 nucleotides, or from about 10 to about 15 nucleotides in length. For example, the effector domain may be 8, 9, 10, 11, 12, 13, 14, 15, 16, 17, 18, 19, or 20 nucleotides in length.

The insertion of point mutations into the U1 domain, i.e., diverging from the consensus sequence SEQ ID NO: 1, SEQ ID NO: 4, or SEQ ID NO: 5, can moderate silencing. Indeed, altering the consensus sequence will produce U1 domains of different strength and affinity for the U1 snRNA, thereby leading to different levels of silencing. Therefore, once an annealing domain has been determined for a gene of interest, different U1 domains of different strength can be attached to the annealing domain to effect different levels of silencing of the gene of interest. For example, gAGGUAAGUA (SEQ ID NO: 3) would bind more weakly to U1 snRNP than SEQ ID NO: 1 and, therefore, would produce a lower level of silencing. As discussed above, nucleotide analogues can be included in the U1 domain to increase the affinity to endogenous U1 snRNP. The addition of nucleotide analogs may not be considered a point mutation if the nucleotide analog binds the same nucleotide as the replaced nucleotide.

The U1AO may be modified to be resistant to nucleases. In a particular embodiment, the U1AO may comprise at least one non-natural nucleotide and/or nucleotide analog. The nucleotide analogs may be used to increase annealing affinity, specificity, bioavailability in the cell and organism, cellular and/or nuclear transport, stability, and/or resistance to degradation. For example, it has been well-established that inclusion of Locked Nucleic Acid (LNA) bases within an oligonucleotide increases the affinity and specificity of annealing of the oligonucleotide to its target site (Kauppinen et al. (2005) Drug Discov. Today Tech., 2:287-290; Orum et al. (2004) Letters Peptide Sci., 10:325-334). Unlike RNAi and RNase H-based silencing technologies, U1AO inhibition does not involve enzymatic activity. As such, there is significantly greater flexibility in the permissible nucleotide analogs that can be employed in the U1AO when compared with oligos for RNAi and RNase H-based silencing technologies.

Nucleotide analogs include, without limitation, nucleotides with phosphate modifications comprising one or more phosphorothioate, phosphorodithioate, phosphodiester, methyl phosphonate, phosphoramidate, methylphosphonate, phosphotriester, phosphoroaridate, morpholino, amidate carbamate, carboxymethyl, acetamidate, polyamide, sulfonate, sulfonamide, sulfamate, formacetal, thioformacetal, and/or alkylsilyl substitutions (see, e.g., Hunziker and Leumann (1995) Nucleic Acid Analogues: Synthesis and Properties, in Modern Synthetic Methods, VCH, 331-417; Mesmaeker et al. (1994) Novel Backbone Replacements for Oligonucleotides, in Carbohydrate Modifications in Antisense Research, ACS, 24-39); nucleotides with modified sugars (see, e.g., U.S. Patent Application Publication No. 2005/0118605) and sugar modifications such as 2′-O-methyl (2′-O-methylnucleotides), 2′-O-methyloxyethoxy, and 2′-halo (e.g., 2′-fluoro); and nucleotide mimetics such as, without limitation, peptide nucleic acids (PNA), morpholino nucleic acids, cyclohexenyl nucleic acids, anhydrohexitol nucleic acids, glycol nucleic acid, threose nucleic acid, and locked nucleic acids (LNA) (see, e.g., U.S. Patent Application Publication No. 2005/0118605). Other nucleotide modifications are also provided in U.S. Pat. Nos. 5,886,165; 6,140,482; 5,693,773; 5,856,462; 5,973,136; 5,929,226; 6,194,598; 6,172,209; 6,175,004; 6,166,197; 6,166,188; 6,160,152; 6,160,109; 6,153,737; 6,147,200; 6,146,829; 6,127,533; and 6,124,445. In a particular embodiment, the U1AO comprises at least one locked nucleic acid. In a particular embodiment, the annealing domain comprises at least one locked nucleic acid (optionally where the effector domain does not contain a locked nucleic acid). In a particular embodiment, the U1AO, particularly the annealing domain, has locked nucleic acids spaced apart by 2-4 nucleotides, particularly three nucleotides.

Notably, care should be taken so as to not design a U1 adaptor wherein the effector domain has significant affinity for the target site of the mRNA or the sites immediately flanking the target site. In other words, the target site should be selected so as to minimize the base pairing potential of the effector domain with the target pre-mRNA, especially the portion flanking upstream of the annealing site.

To increase the silencing ability of the U1AO, the U1AO should also be designed to have low self annealing so as to prevent the formation of hairpins within a single U1 adaptor and/or the formation of homodimers or homopolymers between two or more U1 adaptors.

The annealing and effector domains of the U1AO may be linked such that the effector domain is at the 5′ end and/or 3′ end of the annealing domain. Further, the annealing and effector domains may be operably linked via a linker domain. The linker domain may comprise, for example, 1, 2, 3, 4, 5, 6, 7, 8, 9, 10, up to 15, up to 20, or up to 25 nucleotides.

The U1AO may comprise ribonucleotides and/or deoxynucleotides. With regard to the sequences provided herein, uracil bases and thymidine bases may be exchanged. In a particular embodiment, the U1AO comprises 2′-O-methyl nucleotides, 2′-O-methyloxyethoxy nucleotides, 2′-halo (e.g., 2′-fluoro), and/or locked nucleic acids. In a particular embodiment, the U1AO comprises 2′-O-methyl nucleotides. In a particular embodiment, the U1AO comprises phosphorothioates (e.g., phosphorothioates may be spaced apart by 2-4 nucleotides, particularly three nucleotides).

In a particular embodiment, the U1AO comprises a U1AO provided in FIG. 7A-7B or 8A-8C (particularly in RNA). In a particular embodiment, the U1AO comprises:

(SEQ ID NO: 9)
AUCCCAAUGACACCACAAGCGCCAGGUAAGUAU
or
(SEQ ID NO: 10)
UGCCCAAGUCAUCUCGUUUCGCCAGGUAAGUAU.

In another embodiment, the U1AO has at least 70%, at least 75%, at least 80%, at least 85%, and more particularly at least 90%, at least 95%, at least 97% or more identity with a U1AO sequence provided in FIG. 7A-7B or 8A-8C or SEQ ID NO: 9 or SEQ ID NO: 10, particularly SEQ ID NO: 9. With regard to the sequences provided herein, uracil bases and thymidine bases may be exchanged. In a particular embodiment, the U1AO comprises at least one or all nucleotide analogs. In a particular embodiment, the U1AO comprises 2′-O-methyl nucleotides, 2′-O-methyloxyethoxy nucleotides, 2′-halo (e.g., 2′-fluoro), and/or locked nucleic acids. In a particular embodiment, the U1AO comprises 2′-O-methyl nucleotides. In a particular embodiment, the U1AO comprises phosphorothioates (e.g., phosphorothioates may be spaced apart by 2-4 nucleotides, particularly three nucleotides). In a particular embodiment, the U1AO are modified as set forth in the Example.

In another embodiment of the instant invention, more than one U1AO directed to a gene of interest (SOD) may be used to modulate expression. Multiple U1AO targeting (annealing) to different sequences in the same pre-mRNA can provide enhanced inhibition. Compositions of the instant invention may comprise more than one U1AO directed to the SOD gene (e.g., different targets within the SOD gene).

In still another embodiment, the U1AO can be combined with other methods of modulating the expression of a gene of interest. For example, a U1AO can be used in coordination with other inhibitory nucleic acid molecules such as antisense oligonucleotides or RNase H-based methods, RNAi, miRNA, and morpholino-based methods to give enhanced inhibition. Inasmuch as U1AO utilizes a different mechanism than these other approaches, the combined use will result in an increased inhibition of gene expression compared to the use of a single inhibitory agent alone. Indeed, U1AO may target the biosynthetic step in the nucleus whereas RNAi and certain antisense approaches generally target cytoplasmic stability or translatability of a pre-existing pool of mRNA.

In another aspect of the instant invention, the effector domain of the U1 adaptor can be replaced with the binding site for any one of a number of nuclear factors that regulate gene expression. For example, the binding site for polypyrimidine tract binding protein (PTB) is short and PTB is known to inhibit poly(A) sites. Thus, replacing the effector domain with a high affinity PTB binding site would also silence expression of the target gene.

There are U1 snRNA genes that vary in sequence from the canonical U1 snRNA described hereinabove. Collectively, these U1 snRNA genes can be called the U1 variant genes. Some U1 variant genes are described in GenBank Accession Nos. L78810, AC025268, AC025264 and AL592207 and in Kyriakopoulou et al. (RNA (2006) 12:1603-11), which identified close to 200 potential U1 snRNA-like genes in the human genome. Since some of these U1 variants have a 5′ end sequence different than canonical U1 snRNA, one plausible function is to recognize alternative splice signals during pre-mRNA splicing. Accordingly, the U1 domain of the U1AO of the instant invention may be designed to hybridize with the 5′ end of the U1 variant snRNA in the same way as the U1 domain was designed to hybridize with the canonical U1 snRNA as described herein. The U1AO which hybridize to the U1 variants may then be used to modulate the expression of a gene of interest.

There are many advantages of the U1 adaptor technology to other existing silencing technologies. Certain of these advantages are as follows. First, the U1AO separates into two independent domains: (1) the annealing (i.e., targeting) activity and (2) the inhibitory activity, thereby allowing one to optimize annealing without affecting the inhibitory activity or vice versa. Second, as compared to other technologies, usage of two U1AO to target the same gene gives additive even synergistic inhibition. Third, the U1AO has a novel inhibitory mechanism. Therefore, it will be compatible when used in combination with other methods. Fourth, the U1AO inhibits the biosynthesis of mRNA by inhibiting the critical, nearly-universal, pre-mRNA maturation step of poly(A) tail addition (also called 3′ end processing).

Compositions of the instant invention comprise at least one U1AO of the instant invention and at least one pharmaceutically acceptable carrier. The compositions may further comprise at least one other agent which inhibits the expression of the gene of interest (SOD). For example, the composition may further comprise at least one siRNA or antisense oligonucleotide directed against the gene of interest (SOD).

The U1AO of the present invention may be administered alone, as naked polynucleotides, to cells or an organism, including animals and humans. The U1AO may be administered with an agent which enhances its uptake by cells. In a particular embodiment, the U1AO may be contained within a liposome, nanoparticle, or polymeric composition.

In another embodiment, the U1AO may be delivered to a cell or animal, including humans or canines, in an expression vector such as a plasmid or viral vector. For example, a U1AO can be expressed from a vector such as a plasmid or a virus. Expression of such short RNAs from a plasmid or virus has become routine and can be easily adapted to express a U1AO. Expression vectors for the expression of RNA molecules may employ a strong promoter which may be constitutive or regulated. Such promoters are well known in the art and include, but are not limited to, RNA polymerase II promoters, the T7 RNA polymerase promoter, and the RNA polymerase III promoters U6 and H1. Viral-mediated delivery includes the use of vectors based on, without limitation, retroviruses, adenoviruses, adeno-associated viruses, vaccinia virus, lentiviruses, polioviruses, and herpesviruses.

The pharmaceutical compositions of the present invention can be administered by any suitable route, for example, by injection (e.g., intravenously, intracerebroventricularly, and intramuscularly), by oral, pulmonary, nasal, rectal, or other modes of administration. The compositions can be administered for the treatment of a disease or disorder (e.g., a neurogenerative disease such as DM or ALS) which can be treated through the downregulation of SOD. The compositions may be used in vitro, in vivo, and/or ex vivo. With regard to ex vivo use, the U1AO of the instant invention (or compositions comprising the same) may be delivered to autologous cells (optionally comprising the step of obtaining the cells from the subject) and then re-introduced into the subject. The compositions, U1 AO, and/or vectors of the instant invention may also be comprised in a kit.

The instant invention also encompasses methods of treating, inhibiting (slowing or reducing), and/or preventing a disease or disorder (e.g., a neurogenerative disease such as DM or ALS) in a subject. In a particular embodiment, the methods comprise the administration of a therapeutically effective amount of at least one composition of the instant invention to a subject (e.g., an animal (e.g., canine) or human) in need thereof. In a particular embodiment, the composition comprises at least one U1AO of the instant invention and at least one pharmaceutically acceptable carrier.

The instant methods may further comprise the administration of at least one other agent which inhibits the expression of the target SOD gene. For example, the method may further comprise the administration of at least one siRNA or antisense oligonucleotide directed against the SOD gene. The methods may also comprise the administration of at least one other therapeutic agent (e.g., a symptom-alleviating therapeutic agent for disease or disorder (e.g., a neurogenerative disease such as DM or ALS (e.g., edaravone (Radicava®) and/or riluzole (Rilutek®)))). In a particular embodiment, the therapeutic agent is conjugated to the U1AO (e.g., directly or via a linker; e.g., at the 3′ end and/or 5′end). The therapeutic agent may be administered in separate compositions (e.g., with at least one pharmaceutically acceptable carrier) or in the same composition. The therapeutic agent may be administered simultaneously and/or consecutively with the U1AO.

As stated hereinabove, the U1AO of the present invention may be administered alone (as naked polynucleotides) or may be administered with an agent which enhances its uptake by cells. In a particular embodiment, the U1AO may be contained within a delivery vehicle such as a micelle, liposome, nanoparticle, or polymeric composition. In a particular embodiment, the U1AO is complexed with (e.g., contained within or encapsulated by) a dendrimer, particularly cationic dendrimers such as poly(amido amine) (PAMAM) dendrimers and polypropyleneimine (PPI) dendrimers (e.g., generation 2, 3, 4, or 5). In a particular embodiment, the U1AO is complexed with PPI-G2.

In a particular embodiment, the U1AO are targeted to a particular cell type (e.g., neurons). In a particular embodiment, the U1AO is covalently linked (e.g., directly or via a linker) to at least one targeting moiety. The targeting moiety may be operably linked to the 5′ end, the 3′ end, or both ends or to internal nucleotides. In a particular embodiment, one or more targeting moieties are conjugated to one end of the U1AO (e.g., through a single linker). In a particular embodiment, a complex comprising the U1AO (e.g., a dendrimer, micelle, liposome, nanoparticle, or polymeric composition) is covalently linked (e.g., directly or via a linker) to at least one targeting moiety.

Generally, the linker is a chemical moiety comprising a covalent bond or a chain of atoms that covalently attaches two compounds such as a targeting moiety to the U1AO or complex. The linker can be linked to any synthetically feasible position of the targeting moiety and the U1AO or complex (vehicle). In a particular embodiment, the linker connects the targeting moiety and the U1AO or complex via an amine group and/or sulfhydryl/thiol group, particularly a sulfhydryl/thiol group. For example, the U1AO may be derivatized (e.g., at the 5′ end) with one or more amino or thio groups. In a particular embodiment, the linker is attached at a position which avoids blocking the targeting moiety or the activity of the U1AO. In a particular embodiment, the linker is attached to an internal nucleotide, the 5′ end, and/or 3′ end. Exemplary linkers may comprise at least one optionally substituted; saturated or unsaturated; linear, branched or cyclic alkyl group or an optionally substituted aryl group. The linker may also be a polypeptide (e.g., from about 1 to about 20 amino acids or more, or 1 to about 5). The linker may be biodegradable (cleavable (e.g., comprises a disulfide bond)) under physiological environments or conditions. In a particular embodiment, the linker comprises polyethylene glycol (PEG) (alone or in combination with another linker). In a particular embodiment, the linker is a SPDP (N-Succinimidyl 3-(2-pyridyldithio)-propionate) linker such as LC-SPDP (succinimidyl 6-(3-[2-pyridyldithio]-propionamido)hexanoate) or a SMCC (succinimidyl-4-(N-maleimidomethyl) cyclohexane-1-carboxylate) linker such as LC-SMCC (succinimidyl 4-(N-maleimidomethyl)cyclohexane-1-carboxy-(6-amidocaproate)). The linker may also be non-degradable (non-cleavable) and may be a covalent bond or any other chemical structure which cannot be substantially cleaved or cleaved at all under physiological environments or conditions.

Targeting moieties of the instant invention preferentially bind to the relevant tissue (e.g., nerves) or organ (e.g., brain). In a particular embodiment, the targeting moiety specifically binds to a marker specifically (e.g., only) expressed on the target cells or a marker up-regulated on the target cells compared to other cells. In a particular embodiment, the targeting moiety is an antibody or antibody fragment immunologically specific for a surface protein on the target cells or a surface protein expressed at higher levels (or greater density) on the target cells than other cells, tissues, or organs. The antibody or antibody fragment may be a therapeutic antibody (e.g., possessing a therapeutic effect itself). In a particular embodiment, the targeting moiety is a ligand or binding fragment thereof for a cell surface receptor on the target cells. In a particular embodiment, the targeting moiety is an aptamer.

The U1AO of the instant invention may further be conjugated to other desirable compounds. For example, the U1AO may be further conjugated (directly or via a linker as described above) to detectable agents, therapeutics (e.g., monoclonal antibodies, peptides, proteins, inhibitory nucleic acid molecules, small molecules, chemotherapeutic agents, etc.), carrier protein, and agents which improve bioavailability, stability, and/or absorption (e.g., PEG). The additional compounds may be attached to any synthetically feasible position of the U1AO (or conjugate (e.g., to the U1 Adaptor (e.g., either end or internal nucleotide) or the targeting moiety). Alternatively, the targeting moiety and the U1AO are each individually attached to additional compound (e.g., carrier protein) (as such the additional compound can be considered to serve as the linker between the U1AO and the targeting moiety). In a particular embodiment, the U1AO is conjugated to a targeting moiety (e.g., neuron targeting moiety) at one end and, optionally, a therapeutic agent on the other. Preferentially, the attachment of the additional compounds does not significantly affect the activity of the U1AO or the targeting moiety. Detectable agents may be any compound or protein which may be assayed for directly or indirectly, particularly directly. Detectable agents include, for example, chemiluminescent, bioluminescent, and/or fluorescent compounds or proteins, imaging agent, contrast agent, radionuclides, paramagnetic or superparamagnetic ions, isotopes (e.g., radioisotopes (e.g., ³H (tritium) and ¹⁴C) or stable isotopes (e.g., ²H (deuterium), ¹¹C, ¹³C, ¹⁷O and ¹⁸O), optical agents, and fluorescence agents.

Carrier proteins include, without limitation, albumin, serum albumin (e.g., bovine, human), ovalbumin, and keyhole limpet hemocyanin (KLH). In a particular embodiment, the carrier protein is human serum albumin. Carrier proteins (as well as other proteins or peptides) may be conjugated to the U1AO (or conjugate) at any synthetically feasible position (e.g., either directly or via a linker as described above). For example, linkers (e.g., PEG or LC-SPDP) may be attached to free amino groups found on lysines of the carrier protein and then the U1AO and targeting moieties may be conjugated to the linkers. Any unreacted linkers may be inactivated by blocking with cysteine. In a particular embodiment, the carrier protein (e.g., albumin) is attached to the U1AO via a PEG linker.

The U1AO of the instant invention may be conjugated (e.g., directly or via a linker) to a compound (e.g., antibodies, peptides, proteins, nucleic acid molecules, small molecules, etc.) which targets the U1AO to a desired cell type and/or promotes cellular uptake of the U1AO (e.g., a cell penetrating moiety). The targeting moiety may be operably linked to the 5′ end, the 3′ end, or both ends or to internal nucleotides. In a particular embodiment, the targeting moiety and/or cell penetrating moiety are conjugated to the 5′ end and/or 3′ end. In a particular embodiment, the targeting moiety and/or cell penetrating moiety is conjugated to the 5′ end. In a particular embodiment, the U1AO is conjugated to both a targeting moiety and a cell penetrating moiety. As used herein, the term “cell penetrating agent” or “cell penetrating moiety” refers to compounds or functional groups which mediate transfer of a compound from an extracellular space to within a cell. In a particular embodiment, the U1AO is conjugated to an aptamer. The aptamer may be targeted to a surface compound or protein (e.g., receptor) of a desired cell type (e.g., the surface compound or protein may be preferentially or exclusively expressed on the surface of the cell type to be targeted). In a particular embodiment, the aptamer is a cell penetrating aptamer (e.g., Cl or Otter (see, e.g., Burke, D. H. (2012) Mol. Ther., 20: 251-253)). In a particular embodiment, the U1AO is conjugated to a cell penetrating peptide (e.g., Tat peptides (e.g., YGRKKKRRQRRRPPQ; SEQ ID NO: 6 (optionally acetylated on N-terminus)), Penetratin (e.g., RQIKIWFQNRRMKWKKGG; SEQ ID NO: 7), short amphipathic peptides (e.g., from the Pep- and MPG-families), oligoarginine (e.g., 4-12 consecutive arginine), oligolysine (e.g., 4-12 consecutive lysine)). In a particular embodiment, the U1AO is conjugated to a small molecule such as biotin (as part of targeting antibodies) or a non-polar fluorescent group (e.g., a cyanine such as Cy3 or Cy5) or to other cell penetrating agents.

In a particular embodiment, at least one of the 3′ end and 5′ end of the U1AO comprises a free-SH group.

The U1AO (including the vehicles comprising the same) described herein will generally be administered to a patient as a pharmaceutical preparation. The terms “patient” and “subject”, as used herein, include humans and animals. These U1 adaptors may be employed therapeutically, under the guidance of a physician or veterinarian.

The compositions comprising the U1AO of the instant invention may be conveniently formulated for administration with any pharmaceutically acceptable carrier(s). For example, the U1AO may be formulated with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of the U1AO in the chosen medium may be varied and the medium may be chosen based on the desired route of administration of the pharmaceutical preparation. Except insofar as any conventional media or agent is incompatible with the U1AO to be administered, its use in the pharmaceutical preparation is contemplated.

The dose and dosage regimen of U1AO according to the invention that are suitable for administration to a particular subject may be determined by a physician or veterinarian considering the subject's age, sex, weight, general medical condition, and the specific condition for which the U1AO is being administered and the severity thereof. The physician or veterinarian may also take into account the route of administration, the pharmaceutical carrier, and the U1 AO's biological activity.

Selection of a suitable pharmaceutical preparation will also depend upon the mode of administration chosen. For example, the U1AO of the invention may be administered by direct injection to a desired site (e.g., brain). In this instance, a pharmaceutical preparation comprises the U1AO dispersed in a medium that is compatible with the site of injection. U1AO of the instant invention may be administered by any method. For example, the U1AO of the instant invention can be administered, without limitation parenterally, subcutaneously, orally, topically, pulmonarily, rectally, vaginally, intravenously, intracerebroventricularly, intracranially, intraperitoneally, intrathecally, intracerebrally, epidurally, intramuscularly, intradermally, or intracarotidly. In a particular embodiment, the method of administration is by direct injection (e.g., into the CNS, spine or brain) or intrathecally. Pharmaceutical preparations for injection are known in the art. If injection is selected as a method for administering the U1AO, steps should be taken to ensure that sufficient amounts of the molecules or cells reach their target cells to exert a biological effect.

Pharmaceutical compositions containing a U1AO of the present invention as the active ingredient in intimate admixture with a pharmaceutically acceptable carrier can be prepared according to conventional pharmaceutical compounding techniques. The carrier may take a wide variety of forms depending on the form of preparation desired for administration, e.g., intravenous, oral, direct injection, intracranial, intracerebroventricular, intrathecal, and intravitreal.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation of a particular pathological condition may be determined by dosage concentration curve calculations, as known in the art.

In accordance with the present invention, the appropriate dosage unit for the administration of U1AO may be determined by evaluating the toxicity of the molecules or cells in animal models. Various concentrations of U1AO in pharmaceutical preparations may be administered to mice, and the minimal and maximal dosages may be determined based on the beneficial results and side effects observed as a result of the treatment. Appropriate dosage unit may also be determined by assessing the efficacy of the U1AO treatment in combination with other standard drugs. The dosage units of U1AO may be determined individually or in combination with each treatment according to the effect detected.

The pharmaceutical preparation comprising the U1AO may be administered at appropriate intervals, for example, at least twice a day or more until the pathological symptoms are reduced or alleviated, after which the dosage may be reduced to a maintenance level. The appropriate interval in a particular case would normally depend on the condition of the patient.

### Definitions

The singular forms “a,” “an,” and “the” include plural referents unless the context clearly dictates otherwise.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” may refer to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

A “vector” is a genetic element, such as a plasmid, cosmid, bacmid, phage or virus, to which another genetic sequence or element (either DNA or RNA) may be attached. The vector may be a replicon so as to bring about the replication of the attached sequence or element.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a nucleic acid or a polypeptide coding sequence in a host cell or organism. An “expression vector” is a vector which facilitates the expression of a nucleic acid or a polypeptide coding sequence in a host cell or organism.

The term “oligonucleotide,” as used herein, refers to nucleic acid sequences, primers, and probes of the present invention, and is defined as a nucleic acid molecule comprised of two or more ribo or deoxyribonucleotides, preferably more than three. The exact size of the oligonucleotide will depend on various factors and on the particular application and use of the oligonucleotide.

The phrase “small, interfering RNA (siRNA)” refers to a short (typically less than 30 nucleotides long, more typically between about 21 to about 25 nucleotides in length) double stranded RNA molecule. Typically, the siRNA modulates the expression of a gene to which the siRNA is targeted. The term “short hairpin RNA” or “shRNA” refers to an siRNA precursor that is a single RNA molecule folded into a hairpin structure comprising an siRNA and a single stranded loop portion of at least one, typically 1-10, nucleotide.

The term “RNA interference” or “RNAi” refers generally to a sequence-specific or selective process by which a target molecule (e.g., a target gene, protein or RNA) is downregulated via a double-stranded RNA. The double-stranded RNA structures that typically drive RNAi activity are siRNAs, shRNAs, microRNAs, and other double-stranded structures that can be processed to yield a small RNA species that inhibits expression of a target transcript by RNA interference.

The term “antisense” refers to an oligonucleotide having a sequence that hybridizes to a target sequence in an RNA by Watson-Crick base pairing, to form an RNA:oligonucleotide heteroduplex with the target sequence, typically with an mRNA. The antisense oligonucleotide may have exact sequence complementarity to the target sequence or near complementarity. These antisense oligonucleotides may block or inhibit translation of the mRNA, and/or modify the processing of an mRNA to produce a splice variant of the mRNA. Antisense oligonucleotides are typically between about 5 to about 100 nucleotides in length, more typically, between about 7 and about 50 nucleotides in length, and even more typically between about 10 nucleotides and about 30 nucleotides in length.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like).

The term “isolated” may refer to a compound or complex that has been sufficiently separated from other compounds with which it would naturally be associated. “Isolated” is not meant to exclude artificial or synthetic mixtures with other compounds or materials, or the presence of impurities that do not interfere with fundamental activity or ensuing assays, and that may be present, for example, due to incomplete purification, or the addition of stabilizers.

The term “gene” refers to a nucleic acid comprising an open reading frame encoding a polypeptide, including both exon and (optionally) intron sequences. The nucleic acid may also optionally include non coding sequences such as promoter or enhancer sequences. The term “intron” refers to a DNA sequence present in a given gene that is not translated into protein and is generally found between exons.

As used herein, the term “aptamer” refers to a nucleic acid that specifically binds to a target, such as a protein, through interactions other than Watson-Crick base pairing. In a particular embodiment, the aptamer specifically binds to one or more targets (e.g., a protein or protein complex) to the general exclusion of other molecules in a sample. The aptamer may be a nucleic acid such as an RNA, a DNA, a modified nucleic acid, or a mixture thereof. The aptamer may also be a nucleic acid in a linear or circular form and may be single stranded or double stranded. The aptamer may comprise oligonucleotides that are at least 5, at least 10, at least 15, at least 20, at least 25, at least 30, at least 35, at least 40 or more nucleotides in length. Aptamers may comprise sequences that are up to 40, up to 60, up to 80, up to 100, up to 150, up to 200 or more nucleotides in length. Aptamers may be from about 5 to about 150 nucleotides, from about 10 to about 100 nucleotides, or from about 20 to about 75 nucleotides in length. While aptamers are discussed herein as nucleic acid molecules (e.g., oligonucleotides) aptamers, aptamer equivalents may also be used in place of the nucleic acid aptamers, such as peptide aptamers.

The phrase “operably linked”, as used herein, may refer to a nucleic acid sequence placed into a functional relationship with another nucleic acid sequence. Examples of nucleic acid sequences that may be operably linked include, without limitation, promoters, transcription terminators, enhancers or activators and heterologous genes which when transcribed and, if appropriate to, translated will produce a functional product such as a protein, ribozyme or RNA molecule.

“Pharmaceutically acceptable” indicates approval by a regulatory agency of the Federal government or a state government. “Pharmaceutically acceptable” agents may be listed in the U.S. Pharmacopeia or other generally recognized pharmacopeia for use in animals, and more particularly in humans.

A “carrier” refers to, for example, a diluent, preservative, solubilizer, emulsifier, adjuvant, excipient, auxiliary agent or vehicle with which an active agent of the present invention is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions may be employed as carriers. Suitable pharmaceutical carriers are described, for example, in “Remington's Pharmaceutical Sciences” by E. W. Martin.

An “antibody” or “antibody molecule” is any immunoglobulin, including antibodies and fragments thereof (e.g., immunologically specific fragments), that binds to a specific antigen. As used herein, antibody or antibody molecule contemplates intact immunoglobulin molecules, immunologically active portions of an immunoglobulin molecule, and fusions of immunologically active portions of an immunoglobulin molecule. The term includes polyclonal, monoclonal, chimeric, single domain (Dab) and bispecific antibodies. As used herein, antibody or antibody molecule contemplates recombinantly generated intact immunoglobulin molecules and immunologically active portions of an immunoglobulin molecule such as, without limitation: Fab, Fab′, F(ab′)₂, F(v), scFv, scFv₂, and scFv-Fc.

With respect to antibodies, the term “immunologically specific” refers to antibodies that bind to one or more epitopes of a protein or compound of interest, but which do not substantially recognize and bind other molecules in a sample containing a mixed population of antigenic biological molecules.

The term “treat” refers to the ability of the compound to relieve, alleviate, and/or slow the progression of the patient's disease. In other words, the term “treat” refers to inhibiting and/or reversing the progression of a disease.

The following examples describe illustrative methods of practicing the instant invention and are not intended to limit the scope of the invention in any way.

### EXAMPLE 1

Here, U1 Adaptor Oligonucleotide (U1AO) technology was employed to silence central nervous system (CNS) SOD1 expression in dogs following intrathecal administration.

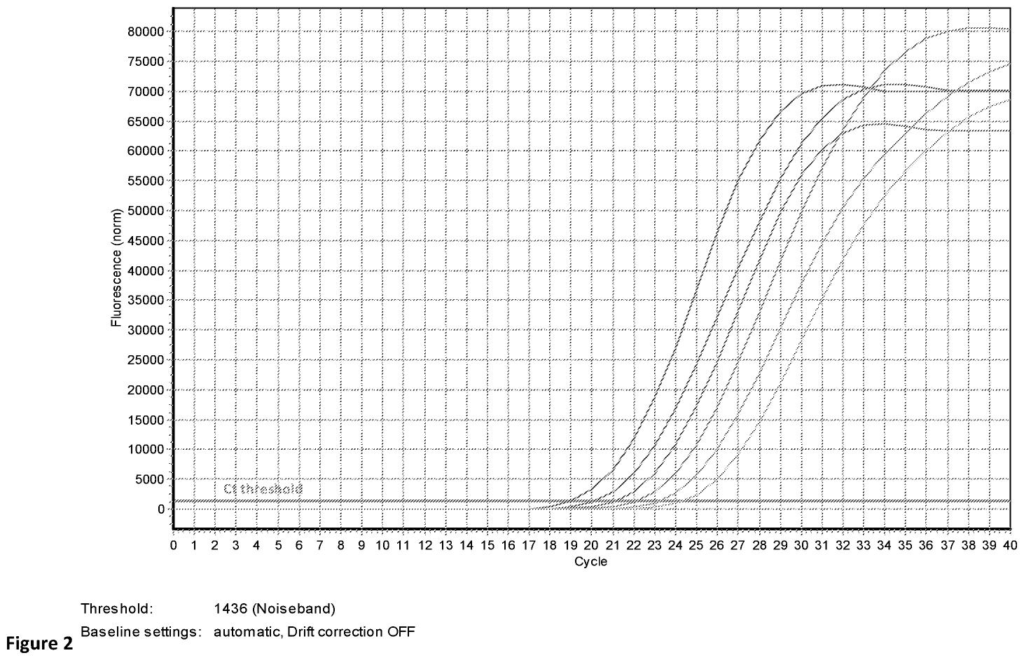

Preliminary in vitro work was performed to identify a potent anti-SOD1 U1AO. Madin-Darby Canine Kidney (MDCK) cells were used for the in vitro studies. MDCK cells are a widely-used immortalized canine dog cell line derived from the kidney of a normal adult female cocker spaniel. FIG. 2 provides a quantitative reverse transcription PCR (RT-qPCR) analysis of MDCK cells for canine SOD1. The results presented in FIG. 2 show that the mRNA for SOD1 is expressed in MDCK cells at a level sufficient to be able to detect silencing of SOD1, and the RT-qPCR methods provide a quantitative assessment of the expression level of the SOD1 gene.

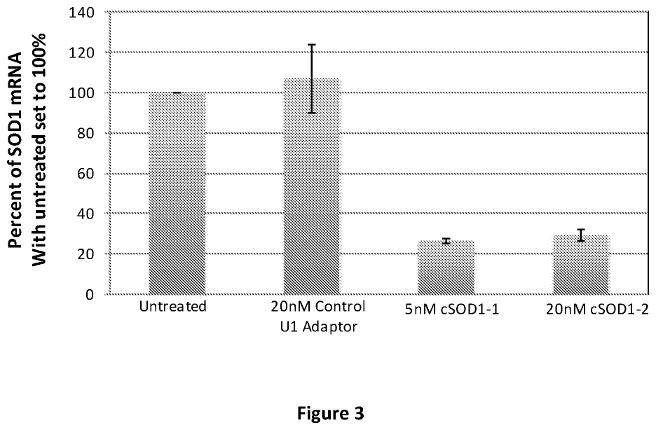

FIG. 3 shows that two anti-SOD1 U1AOs silence canine SOD1 expression down to about 25% as compared to untreated (set to 100%) and control treated MDCK cells. Briefly, 2×10⁵ MDCK cells were mock-transfected or transfected with 20 nM control U1AO, 5 nM cSOD1-1, or 20 nM cSOD1-2. 24 hours post-transfection, an RT-qPCR analysis was performed for canine SOD1 mRNA expression. Expression was normalized to canine HPRT1. As seen in FIG. 3, cSOD1-1 was more potent/effective than cSOD1-2.

The sequence of the cSOD1-1 U1AO is:

(SEQ ID NO: 9)
AUCCCAAUGACACCACAAGCGCCAGGUAAGUAU,

wherein all are RNA bases with 2′O-methyl modification.

The sequence of the cSOD1-2 U1AO is: UGCCCAAGUCAUCUCGUUUCGCCAGGUAAGUAU-(PEG₁₂)-Albumin (SEQ ID NO: 10), wherein all are RNA bases with 2′O-methyl modification.

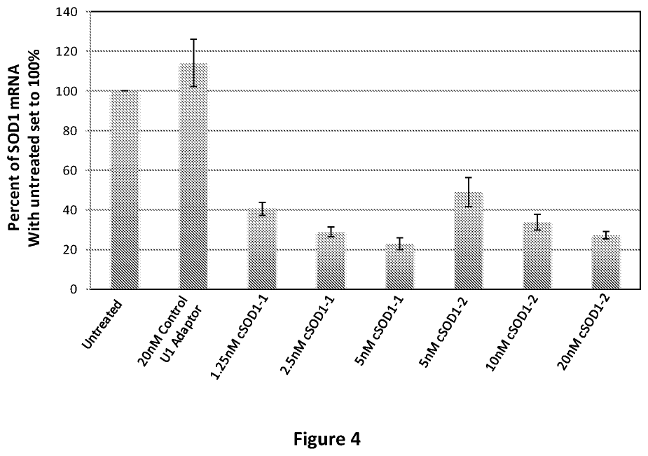

FIG. 4 provides a dose response graph with cSOD1-1 or cSOD1-2. Briefly, MDCK cells were mock-transfected or transfected with 20 nM control U1AO or various amounts of cSOD1-1 or cSOD1-2. 24 hours post-transfection, an RT-qPCR analysis was performed for canine SOD1 mRNA expression. Expression was normalized to canine HPRT1 and untreated expression was set to 100%. As seen in FIG. 4, increasing the amount of either cSOD1-1 or cSOD1-2 resulted in a dose dependent increase in silencing of canine SOD1 mRNA.

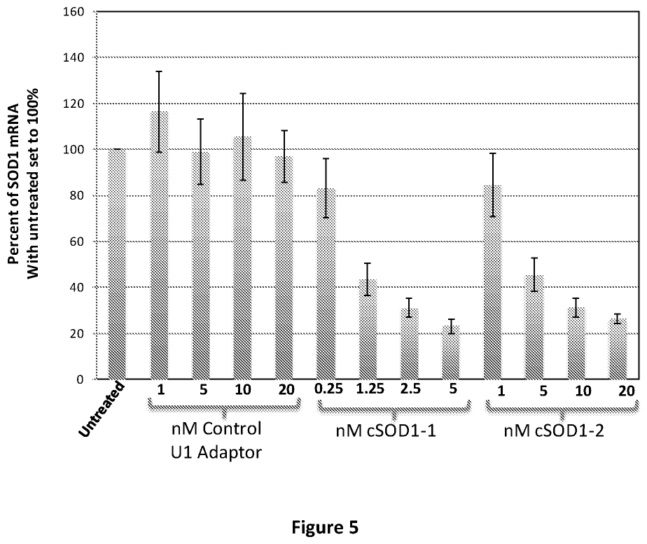

FIG. 5 provides a further dose response graph with cSOD1-1 or cSOD1-2. Briefly, MDCK cells were mock-transfected or transfected with various amounts of control U1AO, cSOD1-1, or cSOD1-2. 24 hours post-transfection, an RT-qPCR analysis was performed for canine SOD1 mRNA expression. Expression was normalized to canine HPRT1 and untreated expression was set to 100%. As seen in FIG. 5, decreasing the amount of either cSOD1-1 or cSOD1-2 eventually resulted in a loss of significant silencing of canine SOD1 mRNA.

Based on the data provided in FIGS. 3-5, cSOD1-1 was selected for in vivo analysis. with phosphorothioate groups and re-named anti-SOD1 U1AO to distinguish from the original cSOD1-1. Anti-SOD1 U1AO was manufactured in appropriate quantities and purity, certified endotoxin-free and then used in dogs as described below. The addition of phosphorothioate increases nuclease resistance when used in vivo. The sequence of the anti-SOD1 U1AO is: A*U*CCCA*AUGA*CACC*ACAA*GCGC*CAGG*UAAG*U*A*U (SEQ ID NO: 9), wherein all are RNA bases with 2′O-methyl modification and * indicates phosphorothioate. The U1AO may be conjugated to -(PEG₁₂)-Albumin at its 3′ end.

Six young, healthy beagles (3 male, 3 female) weighing between 6 and 8 kg underwent physical and neurological examinations and had a routine serum biochemistry panel and complete blood cell count performed. Two dogs were evaluated at a time, with full data analysis prior to moving to the next 2, to ensure dose and protocol adjustment could be made based on results. Dogs were anesthetized, lumbar puncture was performed at the lumbar 5/6 interspace and a cerebrospinal fluid (CSF) sample was taken for a routine CSF analysis. Dogs were sacrificed 5 days later.

In the first 2 dogs, pediatric epidural catheters were placed intrathecally from the lumbar puncture site for slow infusion of vehicle in one dog and 10 mg of anti-SOD1 U1AO in the other dog. In both dogs the volume infused was 2 mL with an additional 0.6 mL lost in the injection apparatus. Due to issues with kinking of these tiny catheters, they were withdrawn and the injections were made from a 20 gauge spinal needle over a period of 10 minutes. The dogs were recovered from anesthesia, and returned to their run. They were evaluated twice a day (physical and neurological examination), blood work was repeated after 2 days and again on day 5 at time of humane euthanasia. Regions of the lumbar, thoracic and cervical spinal cord and the brain were immediately dissected and placed in RNAlater® RNA Stabilization Solution (Invitrogen; Waltham, Mass.). Samples of the liver, kidney and spleen were also obtained. Frozen samples were later analyzed by RT-qPCR for SOD1 and hypoxanthine phosphoribosyltransferase 1 (HPRT1; a housekeeping gene). The rest of the tissues were placed in 10% formalin, trimmed for embedding in paraffin after 3-5 days, sectioned, and stained with hematoxylin and eosin for histopathological examination. These 2 dogs suffered no noticeable effects of the procedure clinically, in their bloodwork or histopathology. When compared with the control dog the level of SOD1 expression in the spinal cord was reduced to 45% but there was no change in expression in the brain.

In the next 2 dogs, the same protocol was repeated (1 control and 1 U1AO) but the administration was refined to allow more efficient flushing of the U1AO from the syringe and catheter intrathecally. Briefly, a 20 gauge spinal needle was attached to a short giving set, which was in turn attached to a filter and 3-way tap. The U1AO was placed in one syringe attached to the 3-way tap and a phosphate buffered saline was placed in another syringe attached to the 3-way tap. The phosphate buffered saline allowed for the flushing of all therapeutic into the intrathecal space. Following infusion, the dog was tilted about 30° for 15 minutes with its head down to allow gravity to spread the drug along the neuraxis. This technique meant that each dog received 2.6 mL (13 mg U1AO in the dog receiving the therapeutic). Once again, the procedure was extremely well tolerated with no adverse effects and the new injection protocol resulted in improved silencing: to 35% expression levels along the spinal cord, 55% in the cerebellum and 74% in the hippocampus.

Given these results, the final 2 dogs were both administered 10 mg of anti-SOD1 U1AO using the improved protocol. Both suffered no adverse events and histopathology was normal. The level of SOD1 silencing achieved was comparable to the previous dogs using the improved methods.

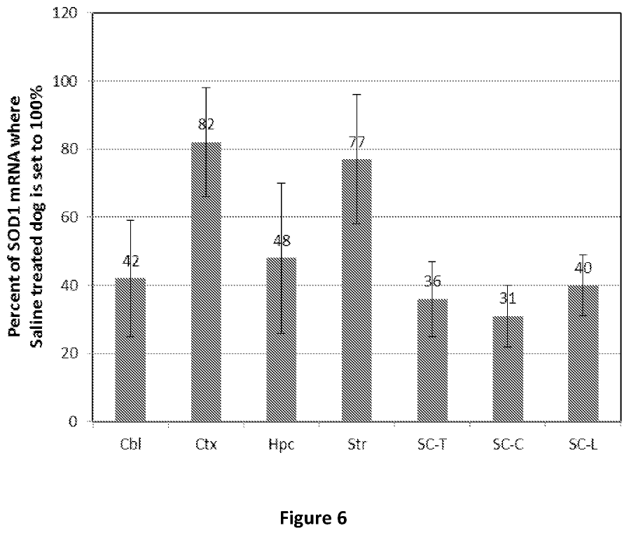

FIG. 6 summarizes the SOD1 expression data from all dogs treated with anti-SOD1 U1AO. Mean SOD1 silencing along the spinal cord ranged from 30-40%, with a gradual decrease in level of silencing moving rostrally in the neuraxis, to 80% in the parietal cortex. Because the pathology is focused primarily on the spinal cord (Braund, et al., Am. J. Vet. Res. (1978) 39(8):1309-1315; Griffiths, et al., J. Small Anim. Pract. (1975) 16(8):461-471; March, et al., (2009) Vet Pathol. 46(2):241-250) and secondarily on the brainstem nuclei (Johnston, et al., Vet. Rec. (2000) 146(22):629-633), clinically relevant silencing of SOD1 has been produced.

### EXAMPLE 2

A prospective phase 1 clinical trial was performed to establish whether the anti-SOD1 U1AO used in FIG. 6 can be administered safely to pet-owned dogs with naturally occurring degenerative myelopathy. The trial comprised treatment with anti-SOD1 U1AO administered intrathecally (IT) on a monthly basis with the option to change frequency to once every two months IT with double the dose. The overall aim was to determine the safety of this treatment by monitoring clinical signs, blood work and CSF parameters as well as overt changes and monitoring of disease progression.

The study comprised four dogs that were recruited based on the following parameters. Dogs were >8 years old, paraparetic with no evidence of focal myelopathy clinically (no spinal pain or cutaneous trunci reflex cut off), had normal blood work and chest radiographs, and tested positive for the SOD1 mutation associated with degenerative myelopathy. Dogs also underwent MRI and CSF analysis to ensure they did not have concurrent spinal neoplasia, myelitis or intervertebral disc disease that could present with similar clinical signs. Furthermore, each owner had to be willing to give several days a month of time to check the dog into the hospital the day before the IT procedure, be at the hospital during the procedure, and then the day after the procedure check the dog out of the hospital.

The study endpoint was when owners elected to either withdraw due to personal reasons or euthanasia of the dog for either personal or compassionate reasons due to disease progression. The owners bore no costs for any aspects of the trial including hospital stay, procedure, analysis or the anti-SOD1 U1AO test compound.

Throughout the study, dogs were monitored once a month by physical and neurological examination, serum biochemistry panel, complete blood cell count and CSF analysis for evidence of adverse effects. Their neurological function was assessed once a month using established ordinal gait and proprioception methodologies. In the event the owner elects euthanasia for personal or compassionate reasons due to disease progression, the tissues and serum were to be analyzed to obtain PK/PD data that includes: 1) quantification of SOD1 expression using RT-qPCR (saline dog from FIG. 6 was set to 100%) and immunohistochemistry and 2) determination of the amount of anti-SOD1 U1AO in the tissues and serum using Northern blot analysis.

For monthly dosing, the amount administered was 1.5 mg anti-SOD1 U1AO/kg of body weight. For dosing once every two months, the dose was doubled to 3.0 mg anti-SOD1 U1AO/kg of body weight. The IT procedure followed the protocol described in Example 1.

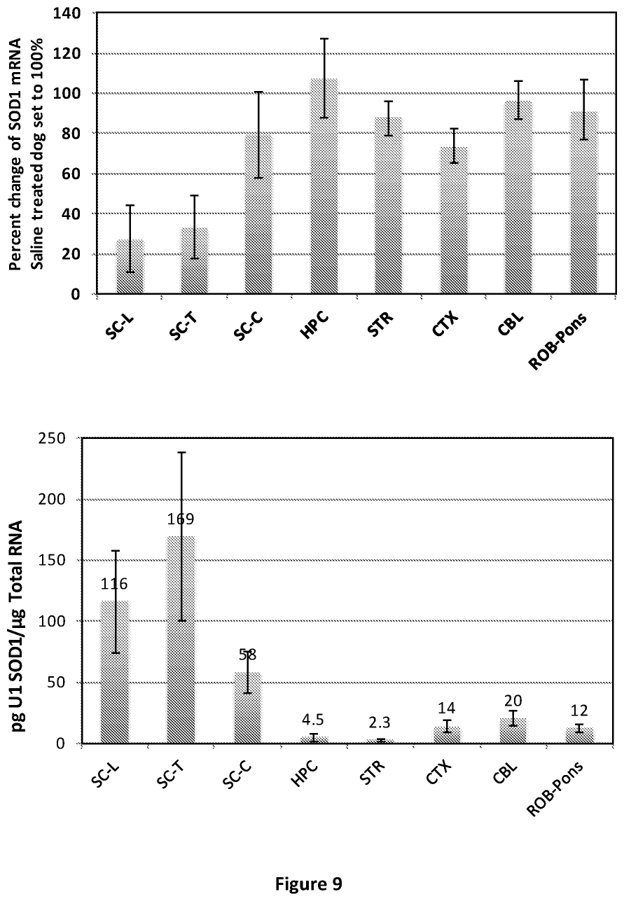

Initially, two dogs with DM were recruited and are called Dog-73A and Dog-73B. Dog-73A (˜11 kg, Corgy, male) underwent six monthly treatments followed by two month interval treatments with a double dose. For personal reasons, the owner elected that Dog-73A undergo euthanasia, which occurred ˜10 weeks after the final treatment. Tissues were collected and underwent PK/PD analysis with results summarized in FIG. 9.

Dog-73B (˜32 kg, German Shephard, male) underwent six monthly treatments and subsequently has undergone treatments at two month intervals at the double dose until the present time. Dog-73B is now in month #16 of treatment.

For both dogs 73A and 73B, no adverse events were encountered and both dog's neurological signs progressed slowly.

Recruitment of two further dogs called Dog-73E and Dog-73F was delayed relative to Dog-73A and Dog-73B with the aim of generating enough data on dosing and safety to adjust dose rates and intervals, if appropriate.

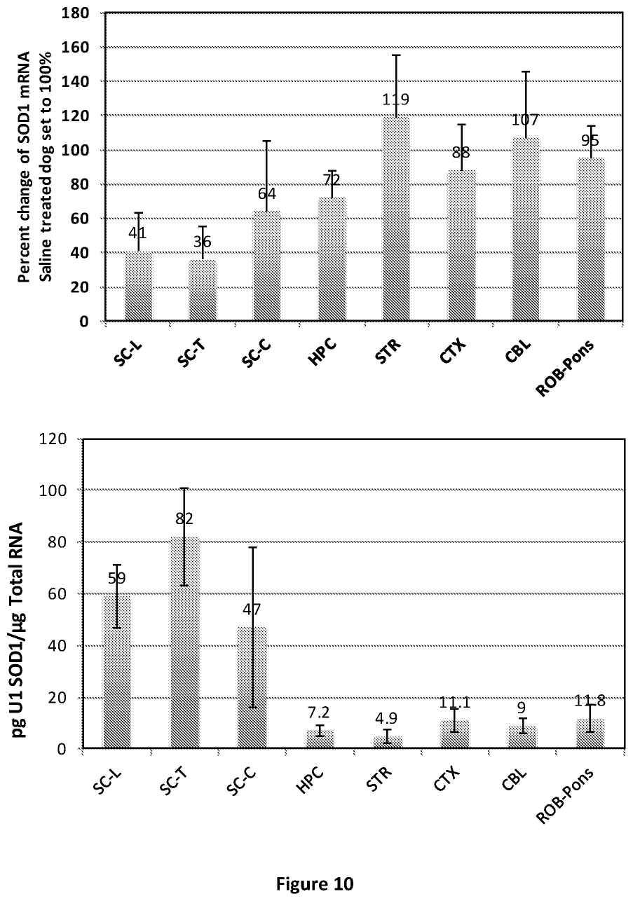

Dog-73E (˜10 kg, Jack Russell mix, female) underwent two 30-day-spaced treatments followed by two double-dose treatments spaced two months apart. For personal reasons, the owner elected that Dog-73E undergo euthanasia, which occurred ˜9 weeks after the final treatment. Tissues were collected and underwent PK/PD analysis with results summarized in FIG. 10.

Dog-73F (˜22 kg, breed not disclosed, male) underwent two monthly-spaced treatments followed by treatments at two month intervals at double dose until the present time. Dog-73F is now in month #9 of treatment.

For all four dogs, no adverse events were observed and the dog's neurological signs progressed slowly. Dog-73B and Dog-73F have remained in stage 2 of the disease in which they need support of their hind quarters to get around and both have shown only slow progression of their clinical signs. The progression of the disease was significantly slower than typical dogs without treatment, which would have been expected to progress to at least stage 3 during this time. Dog-73A and Dog-73E also remained in stage 2 of the disease throughout their treatment periods until euthanasia.

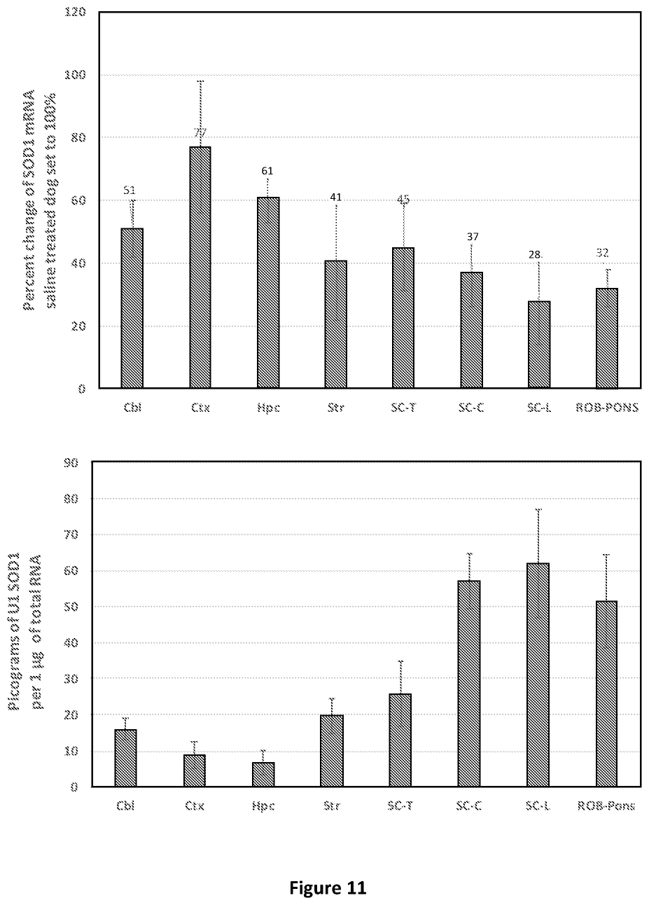

In addition to the above, a separate single dog study, Dog-73C (˜21 kg, breed not disclosed, male), was performed using an end-stage DM dog who was nearly completely paralyzed (stage 4) and whose owners agreed to a single dose PK/PD study with a 5 day duration. In addition to the PK/PD data, this study allowed for the assessment of whether the treatment would be safe to use on an end stage dog. After the single IT treatment at the 1.5 mg/kg body weight dose, no adverse events were encountered and after a 5 day duration, Dog-73C was euthanized, tissues collected with PK/PD results summarized in FIG. 11.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

Several publications and patent documents are cited in the foregoing specification in order to more fully describe the state of the art to which this invention pertains. The disclosure of each of these citations is incorporated by reference herein.

1. A U1 adaptor oligonucleotide comprising a nucleic acid molecule that comprises an annealing domain operably linked to an effector domain, wherein said annealing domain hybridizes to the pre-mRNA of a superoxide dismutase 1 (SOD1) gene, and wherein said effector domain hybridizes to the U1 snRNA of U1 snRNP.

2-4. (canceled)

5. The U1 adaptor oligonucleotide of claim 1, wherein said effector domain comprises a nucleic acid sequence that is at least 75%, at least 80%, at least 85%, at least 90%, at least 95%, at least 97%, or at least 100% identical to the nucleic acid sequence of any one of SEQ ID NO: 1, SEQ ID NO: 4, or SEQ ID NO: 5, wherein uracil bases and thymidine bases may be exchanged.

6. The U1 adaptor oligonucleotide of claim 1, further comprising at least one targeting moiety operably linked to said U1 adaptor oligonucleotide.

7. The U1 adaptor oligonucleotide of claim 1, wherein the nucleic acid sequence of said U1 adaptor oligonucleotide comprises at least one nucleotide analog.

8. The U1 adaptor oligonucleotide of claim 7, wherein the at least one nucleotide analog is selected from the group consisting of a 2′-O-methyl nucleotide, a 2′-O-methyloxyethoxy nucleotide, a 2′-halo nucleotide, a locked nucleic acid, and a phosphorothioate.

9. (canceled)

10. The U1 adaptor oligonucleotide of claim 1, wherein said annealing domain hybridizes with a target sequence in the 3′ terminal exon of the pre-mRNA of the SOD1 gene.

11-20. (canceled)

21. The U1 adaptor oligonucleotide of claim 1, wherein said annealing domain comprises a nucleic acid sequence that is at least 75%, at least 80%, at least 85%, at least 90%, at least 95%, at least 97%, or at least 100% complementary to the sequence of SEQ ID NO: 8 or SEQ ID NO: 11, wherein uracil bases and thymidine bases may be exchanged.

22. The U1 adaptor oligonucleotide of claim 1, wherein said U1 adaptor oligonucleotide comprises a nucleic acid sequence that is at least 75%, at least 80%, at least 85%, at least 90%, at least 95%, at least 97%, or at least 100% identical to SEQ ID NO: 9 or SEQ ID NO: 10, wherein uracil bases and thymidine bases may be exchanged.

23. A pharmaceutical composition comprising the U1 adaptor oligonucleotide of claim 1 and at least one pharmaceutically acceptable carrier.

24. The composition of claim 23, wherein said composition further comprises at least one siRNA or antisense oligonucleotide directed against said SOD1 gene.

25. A method of inhibiting the expression of the SOD1 gene comprising delivering to a cell a U1 adaptor oligonucleotide of claim 1.

26. A method of inhibiting the expression of the SOD1 gene, comprising delivering to a cell a first and second U1 adaptor oligonucleotide of claim 1, wherein the annealing domains of said first and second U1 adaptor oligonucleotides hybridize with different target sequences in said SOD1 gene.

27. A method of treating degenerative myelopathy (DM) or amyotrophic lateral sclerosis (ALS) in a subject in need thereof, said method comprising administering the U1 adaptor oligonucleotide of claim 1 to said subject.

28. A method of treating degenerative myelopathy (DM) or amyotrophic lateral sclerosis (ALS) in a subject in need thereof, said method comprising administering a first and second U1 adaptor oligonucleotide of claim 1, wherein the annealing domains of said first and second U1 adaptor oligonucleotides hybridize with different target sequences in said SOD1 gene.

29. The method of claim 27, further comprising the administration of at least one siRNA or antisense oligonucleotide directed against said SOD1 gene.

30. The U1 adaptor oligonucleotide of claim 1, wherein said U1 adaptor oligonucleotide comprises a nucleic acid sequence that is at least 75%, at least 80%, at least 85%, at least 90%, at least 95%, at least 97%, or at least 100% identical to the sequence of any one of SEQ ID NOS: 94-177.

31. The U1 adaptor oligonucleotide of claim 1, wherein said U1 adaptor oligonucleotide comprises a nucleic acid sequence that is at least 75%, at least 80%, at least 85%, at least 90%, at least 95%, at least 97%, or at least 100% identical to the sequence of any one of SEQ ID NOS: 296-413.

32. The U1 adaptor oligonucleotide of claim 1, wherein said annealing domain comprises a nucleic acid sequence that is at least 75%, at least 80%, at least 85%, at least 90%, at least 95%, at least 97%, or at least 100% complementary to the sequence of any one of SEQ ID NOS: 12-93, wherein uracil bases and thymidine bases may be exchanged.

33. The U1 adaptor oligonucleotide of claim 1, wherein said annealing domain comprises a nucleic acid sequence that is at least 75%, at least 80%, at least 85%, at least 90%, at least 95%, at least 97%, or at least 100% complementary to the sequence of any one of SEQ ID NOS: 178-295, wherein uracil bases and thymidine bases may be exchanged.

34. The U1 adaptor oligonucleotide of claim 1, further comprising at least one cell penetrating moiety operably linked to said U1 adaptor oligonucleotide.
